SEQUENCE LISTING (26 sequences)

SEQ ID NO: 1 - RNA, 41 nt, Artificial Sequence
  Guide RNA CRI-41
uaauuucuacuaaguguagaucgcgccaaagcgacagccac

SEQ ID NO: 2 - RNA, 41 nt, Artificial Sequence
  Guide RNA CRI-45
uaauuucuacuaaguguagaugcuugguuggacuugacgau

SEQ ID NO: 3 - RNA, 41 nt, Artificial Sequence
  Guide RNA CRI-47
uaauuucuacuaaguguagauaguauugccugugucgucgg

SEQ ID NO: 4 - DNA, 45 bp, Artificial Sequence
  dsDNA target CRI-40
ctgcgtggtgctttacgcgccaaagcgacagccacgcatctcgtg

SEQ ID NO: 5 - DNA, 55 bp, Artificial Sequence
  dsDNA target CRI-44
cgcggtgatacgggcaagtattttggcttggttggacttgacgatcatcaggtag

SEQ ID NO: 6 - DNA, 55 bp, Artificial Sequence
  dsDNA target CRI-46
cgtgcctaccgtttcagtattgcctgtgtcgtcggttccagaggtgttcgaccgc

SEQ ID NO: 7 - DNA, 10 bp, Artificial Sequence
  Biotin Alexa488
ttattttatt

SEQ ID NO: 8 - DNA, 1299 bp, Artificial Sequence
  Target segment
atgatctttggtgccgcgctattggggccgcagtgtaggcacgggctatctggagaacgatcgagttggttactgttcgtcgttggtctggagttgcccgtgcctaccgtttcagtattgcctgtgtcgtcggttccagaggtgttcgaccgctggagtgctggtgagcgcagcgagctcatcggatcggagtttctcgatgtgttggcctcggtgccagacccgagggacccacgcggtcggaggtattcgctgatggctttgttggcgatcgcggttctggccactgccgcggggatgcgcggctatgctggttttgccacatgggcggccaccgcttccgatgatgtgttggcccaattaggggtccggttccggcggcccagtgagaagaccttccgcgctgttttgtctcggctagaccccgccgacctcaacgccaggatgggcagttacttcactgcacacgtggccagcagcgaccccagtggattggtgccgatcgcgttggacggcaagatgctgcgtggtgctttacgcgccaaagcgacagccacgcatctcgtgtcggtgttcgcccaccgtgcccgattggtgctcggtcaactcgctgtcgccgagaaaagcaatgaaattccctgcgtacgtgccctgctcacgctgctgccggataacttgcggtggctggtcaccgtggatgcgatgcatacccaggtcgtcaccgcgaagttgatctgcgccaccttgaagtcgcactacctgatgatcgtcaagtccaaccaagccaaaatacttgcccgtatcaccgcgctgccctgggccgaggtgcccgcagccgctaccgacgactcccgcggccacggccgtgtcgagacccgcaccctgcaaatcatcaccgctgcacgaggaatcggcttcccctacgcaaaacaaatcatccggatcactcgtgaacgcttgatcaccgccaccgaccagcgcagcgtggaggtggtctatgccatctgcagcctgccgttcgagcacgcccgccctaccgcgatcatgacctggatgcgtcaacactgcggaatcgagaacagcctgcactggatacgcgacgtcaccttcgacgaagaccgtcacaggccacataccggaaacggcgcacaggtcctagcaacgctacgcaacaccgcgatcaatctgcaccgcctcaacggcgccgacaacatcgccgaagcctgccggatcaccgctttgaccgccaaccgccgcctagacctcctcaatccacaattccccagctcacaagcctgc

SEQ ID NO: 9 - RNA, 41 nt, Artificial Sequence
  Guide RNA CRI-33
uaauuucuacuaaguguagaucuguucgucguuggucugga

SEQ ID NO: 10 - RNA, 41 nt, Artificial Sequence
  Guide RNA CRI-49
uaauuucuacuaaguguagauucgauguguuggccucggug

SEQ ID NO: 11 - RNA, 41 nt, Artificial Sequence
  Guide RNA CRI-90
uaauuucuacuaaguguagaugaccgcuggagugcugguga

SEQ ID NO: 12 - RNA, 41 nt, Artificial Sequence
  Guide RNA CRI-98
uaauuucuacuaaguguagaucaauuccccagcucacaagc

SEQ ID NO: 13 - RNA, 41 nt, Artificial Sequence
  Guide RNA CRI-74
uaauuucuacuaaguguagaugugccgaucgcguuggacgg

SEQ ID NO: 14 - RNA, 41 nt, Artificial Sequence
  Guide RNA CRI-84
uaauuucuacuaaguguagauaggcggugcagauugaucgc

SEQ ID NO: 15 - RNA, 41 nt, Artificial Sequence
  Guide RNA CRI-93
uaauuucuacuaaguguagauacugcacacguggccagcag

SEQ ID NO: 16 - RNA, 41 nt, Artificial Sequence
  Guide RNA CRI-94
uaauuucuacuaaguguagaugcucacgcugcugccggaua

SEQ ID NO: 17 - RNA, 41 nt, Artificial Sequence
  Guide RNAs CRI-58
uaauuucuacuaaguguagauccacaugggcggccaccgcu

SEQ ID NO: 18 - RNA, 41 nt, Artificial Sequence
  Guide RNA CRI-71
uaauuucuacuaaguguagaucgcuauuggggccgcagugu

SEQ ID NO: 19 - RNA, 41 nt, Artificial Sequence
  Guide RNA CRI-73
uaauuucuacuaaguguagaugggguccgguuccggcggcc

SEQ ID NO: 20 - RNA, 41 nt, Artificial Sequence
  Guide RNA CRI-75
uaauuucuacuaaguguagaugcccaccgugcccgauuggu

SEQ ID NO: 21 - RNA, 41 nt, Artificial Sequence
  Guide RNA CRI-77
uaauuucuacuaaguguagauaucugcgccaccuugaaguc

SEQ ID NO: 22 - RNA, 41 nt, Artificial Sequence
  Guide RNA CRI-57
uaauuucuacuaaguguagauuuggcgaucgcgguucuggc

SEQ ID NO: 23 - RNA, 41 nt, Artificial Sequence
  Guide RNA CRI-64
uaauuucuacuaaguguagaucgguauguggccugugacgg

SEQ ID NO: 24 - RNA, 41 nt, Artificial Sequence
  Guide RNA CRI-80
uaauuucuacuaaguguagauucugcagccugccguucgag

SEQ ID NO: 25 - RNA, 41 nt, Artificial Sequence
  Guide RNA CRI-81
uaauuucuacuaaguguagauugcgucaacacugcggaauc

SEQ ID NO: 26 - DNA, 10 bp, Artificial Sequence
  Biotin-C-Alexa488
cccccccccc

(19) United States
(12) Patent Application Publication
(10) Pub. No.: US 2022/0106647 A1
(43) Pub. Date: Apr. 7, 2022

(54) SOLUTION-PHASE, TRANS-ACTIVATED REPORTER SYSTEMS FOR USE IN CRISPR-BASED NUCLEIC ACID SEQUENCE DETECTIONS

(71) Applicant: Tokitae LLC, Bellevue, WA (US)

(72) Inventors: Ted Baughman, Redmond, WA (US); Anne-Laure M. Le Ny, Issaquah, WA (US); Philip Leung, Seattle, WA (US); Damian Madan, Issaquah, WA (US); Eric Nalefski, Bainbridge Island, WA (US)

(73) Assignee: Tokitae LLC, Bellevue, WA (US)

(21) Appl. No.: 17/430,235

(22) PCT Filed: Feb. 7, 2020
(86) PCT No.: PCT/US2020/017169

§ 371 Date: Aug. 11, 2021

Related U.S. Application Data
(60) Provisional application No. 62/803,901, filed on Feb. 11, 2019.

(51) Int. Cl. C12Q 1/6897; C12N 9/22; C12N 15/11

(52) U.S. Cl. CPC C12Q 1/6897; C12N 9/22; C12N 2800/80; C12N 2310/20; C12N 15/11

(57) ABSTRACT

Embodiments disclosed herein include devices, methods, and systems for direct, selective, and sensitive detection of single-stranded and double-stranded target nucleic acid sequences from various sources in a solution-based system. When activated by binding a target nucleic acid sequence, the Cas protein cleaves a tether separating a reporter molecule from a capture moiety. The capture moiety can then be used to remove, localize, or sequester uncleaved molecule containing intact tethers. In some embodiments, the systems, methods, and devices may include a filter, a membrane, or other molecules that may help to separate the tethered and untethered reporter molecules and/or capture the reporter molecules. These devices, systems, and techniques allow a user to rapidly process samples that may contain the target nucleic acid, in some cases, without needing to amplify the target sequences, and without the need for sophisticated or expensive laboratory equipment. These devices and methods may be used to assay a wide variety of samples and target nucleic acid sources, for the presence or absence of a specific target sequences. Compositions and kits, useful in practicing these methods, for example detecting a target RNA or DNA in a biological sample, are also described.

### CROSS-REFERENCE TO RELATED CASES

This application is a U.S. national phase entry of and claims priority to PCT International Phase Application No. PCT/US2020/017169, filed Feb. 7, 2020, which claims priority to U.S. Patent Application No. 62/803,901, filed Feb. 11, 2019. The entire contents of the above-referenced applications and of all priority documents referenced in the Application Data Sheet filed herewith are hereby incorporated by reference for all purposes.

### INCORPORATION-BY-REFERENCE OF SEQUENCE LISTING

The present application is being filed along with a Sequence Listing in electronic format. The Sequence Listing is provided as a file entitled GLOB-2020040pct-US_sequence_2021-12-20.txt, which is 6,945 bytes in size, created and last modified on Dec. 20, 2021. The information in the accompanying Sequence Listing is incorporated by reference in its entirety into this application.

### BACKGROUND

The CRISPR (clustered regulatory interspaced short palindromic repeats) system is a prokaryotic system for recognizing and modifying foreign genetic elements (e.g. plasmids, viruses, phages). Cas (CRISPR-associated) proteins, with the help of RNA sequences, recognize and cut DNA (deoxyribonucleic acid) and/or foreign RNA. This is likely a first step that may result, ultimately, in cell death, limiting spread of the foreign nucleic acid.

Cas 12 or Cas12a, is a programmable DNA endonuclease, guided by a guide RNA, that possesses both specific and non-specific endonuclease (DNase) activity. Activation of Cas12a's DNase activity requires binding a “guide RNA sequence,” which then allows Cas12a to bind a complimentary double-stranded DNA (dsDNA) sequence.

Cas 13 or Cas13a possess non-specific ribonuclease (RNase) activity, which may be dormant until being activated by the binding of other factors to the protein. Specifically, the RNase activity of Cas13a is believed to be activated by recognition of foreign nucleic acid sequences. Like Cas 12, Cas 13 is programmable—that is it may be engineered to detect specific RNA sequences.

Described herein is a solution-based, sensitive, low-cost, rapid, and easy to use system for identification of specific target (or activator) sequences using Cas proteins that may be performed without nucleic acid sequence amplification. The disclosed devices are configured to minimize or reduce steric hindrance of the Cas protein's activity. Use of the disclosed devices greatly enhances the sensitivity and speed of identification compared to other devices, and methods.

### SUMMARY

The disclosed devices, methods, and systems provide for highly sensitive, rapid, sequence-specific, and solution-based detection of target nucleic acid sequences in a biological sample. An indicator device comprises a reporter molecule tethered to a capture moiety by an indicator nucleic acid sequence. The indicator nucleic acid sequence is susceptible to cleavage by a CRISPR nuclease that is activated upon sequence-specific binding to the target nucleic acid. The indicator device is designed to minimize steric hindrance of the nuclease binding the indicator sequence. In an embodiment, a simple pull-down or flow capture process can be used to determine whether the indicator sequence has been cleaved, indicating the presence of the target sequence.

Generally, embodiments of the present disclosure relate to devices, methods, and systems for direct, selective, and sensitive detection of target nucleic acid sequences from various sources. These devices, systems, and techniques allow a user to rapidly process samples that may contain the target nucleic acid sequence, without needing to amplify the target sequences first, or perform sophisticated chemical or biological techniques. Indeed, in various embodiments, the disclosed devices, methods, and systems may be enhanced by amplifying signals through interaction of two or more enzymes, such as RNA nucleases, proteases, peptidases, lipases, glycases, and endonucleases, etc. These devices and methods may be used to assay a wide variety of samples and target nucleic acid sources, for the presence or absence of a specific target nucleic acid sequences. Compositions and kits, useful in practicing these methods, for example detecting a target nucleic acid sequence in a biological sample, are also described.

In an embodiment, a device may aid in determining a presence of a target nucleic acid sequence, for example ssRNA (single-stranded RNA), dsDNA (double stranded DNA) or ssDNA (single-stranded DNA). The device includes at least one reporter molecule, a tether molecule, and a capture moiety. The tether molecule includes a first end, a second end, and at least one indicator nucleic acid sequence for sensing the presence of an activated Cas 12 or Cas13 enzyme, wherein the at least one reporter molecule is attached at the first end, and the capture moiety is attached at the second end. In an embodiment, the disclosed device may include a capture moiety binding molecule (or simply “binding molecule”), the binding molecule for interacting with the capture moiety. In an embodiment, the device may include an assay area and a reporting area

In an embodiment, a method of constructing a device includes synthesizing a tether molecule having a first end, a second end, and at least one indicator nucleic acid (DNA) sequence positioned between the first and second end. In an embodiment, the indicator sequence may include at least two nucleobases, selected from thymine, adenosine, guanine, cytosine, and uracil bases. The method further includes attaching a reporter molecule at the first end of the tether molecule and attaching the second end of the tether molecule to a capture moiety, wherein the capture moiety has at least one measurable dimension less than about 5 nm.

In an embodiment, a system for determining a presence of a target nucleic acid sequence may include a modified Cas12a molecule, including a guide RNA sequence complementary to the target nucleic acid sequence, a device for determining a presence of a nuclease, the device including at least one reporter molecule, a capture moiety, and a tether molecule having a first end, a second end, and at least one indicator nucleic acid sequence positioned between the first and second end, wherein the at least one reporter molecule is attached at the first end, and the capture moiety is attached at the second end of the tether molecule. The device may further include an assay compartment, a detection compartment; and/or a filter positioned between the assay compartment and the detection compartment, wherein the filter is permeable to an untethered reporter molecule.

In an embodiment, a method of detecting a target nucleic acid sequence in a biological sample may include combining the biological sample with a composition to create a sample mixture, the composition including at least one modified Cas molecule including a guide RNA having a sequence complementary to the target nucleic acid sequence. The method further includes incubating the sample mixture with an indicator device in solution to create an assay solution, the indicator device including a capture moiety, at least one reporter molecule, and a tether molecule having a first end and a second end, wherein the tether molecule is attached at a first end to the capture moiety and attached at the second end to the at least one reporter molecule, the tether molecule including at least one indicator nucleic acid sequence positioned between the first and second ends. The method further includes incubating the assay solution for an assay period, incubating the assay solution with a capture moiety binding molecule, and allowing the capture moiety to bind to the binding molecule. Next, a step of applying a separating force to the detection solution, and a step of detecting a signal from an untethered reporter molecule, wherein if the detected signal is greater than a background value, the target nucleic acid sequence is present in the biological sample, wherein the background value is obtained from a biological sample lacking the target nucleic acid sequence.

An embodiment of the disclosed method may be useful in identifying pathogens in a sample obtained from a human or non-human patient, from an environmental sample, an agricultural sample, or a food sample. An embodiment of the disclosed method may be useful in identifying exogenous DNA or RNA, for example microRNA (miRNA) species. In an embodiment, the disclosed methods are able to detect a target nucleic acid sequence in a biological sample without sophisticated laboratory equipment.

Features from any of the disclosed embodiments can be used in combination with one another, without limitation. In addition, other features and advantages of the present disclosure will become apparent to those of ordinary skill in the art through consideration of the following detailed description and the accompanying drawings.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a schematic of an embodiment of the presently disclosed devices. The upper panel shows an indicator device with a capture moiety tethered to reporter molecule, and below the indicator device is a schematic of a binding molecule. The lower panel is a detailed schematic showing construction of an embodiment of the indicator device molecule.

FIG. 2 shows various embodiments of detection devices for use with any of the disclosed methods and systems. Panel A shows a lateral flow embodiment with arrow showing direction of flow. Panel B shows a microfuge tube embodiment.

FIG. 3 is a diagram showing an embodiment of the disclosed device and method. FIG. 3 depicts an embodiment having a first device for detecting the presence of an activated Cas protein, with a reporter molecule tethered to the first device.

FIGS. 4A and 4B are a flow diagram of an embodiment of the disclosed method (FIG. 4A), and a block diagram of an embodiment of the disclosed system (FIG. 4B).

FIG. 5 shows results from Example 1 assessing specificity of Cas/Guide complex to target DNA.

FIG. 6 shows results from Example 2 comparing LOD between soluble and bead substrates.

FIG. 7 shows results from Example 3 using an enzymatic reporter for a soluble assay.

FIG. 8 shows results from Example 4 testing target detection efficiency by varying numbers of guides.

### DEFINITIONS

“Oligonucleotide,” “polynucleotide,” and “nucleic acid,” are used interchangeably herein. These terms may refer to a polymeric form of nucleic acids of any length, strandedness (double or single), and either ribonucleotides (RNA) or deoxyribonucleotides (DNA), and hybrid molecules (comprising DNA and RNA). The disclosed nucleic acids may also include naturally occurring and synthetic or non-natural nucleobases. Natural nucleobases include adenine (A), thymine (T), cytosine (C), guanine (G), and uracil (U).

“Complementarity” refers to a first nucleic acid having a first sequence that allows it to “base pair,” “bind,” “anneal”, or “hybridize,” to a second nucleic acid. In an embodiment, the first nucleic acid may be an RNA sequence and the second may be a single- or double stranded DNA or RNA sequence. In an embodiment, the first nucleic acid may be a DNA sequence and the second may be a single- or double-stranded DNA or RNA sequence. Binding may be affected by the amount of complementarity and certain external conditions such as ionic strength of the environment, temperature, etc. Base-pairing rules are well known in the art (A pairs with T in DNA, and with U in RNA; and G pairs with C). In some cases, RNA may include pairings where G may pair with U. Complementarity does not, in all cases, indicate complete or 100% complementarity. For example, complementarity may be less than 100% and more than about 60%, for example two sequences may be greater than 60% or less than 100% complementary over a given length of sequence (for example greater than 10 nt. and less than about 220 nt).

“Protein,” “peptide,” “polypeptide” are used interchangeably. The terms refer to a polymeric form of amino acids of any length, which may include natural and non-natural residues. The residues may also be modified prior to, or after incorporation into the polypeptide. In some embodiments, the polypeptides may be branched as well as linear.

“Programmed,” in reference to Cas proteins, refers to a Cas protein that includes a guide RNA that contains a sequence complementary to an activator (or target) sequence. Typically, a programmed Cas protein includes an engineered guide RNA.

“Cas protein” is a CRISPR associated protein. The presently disclosed Cas proteins possess a nuclease activity that may be activated upon binding of a target sequence to a guide RNA bound by the Cas protein. As disclosed in more detail below, the guide RNA may, with other sequences, comprise a crRNA, which may, in some embodiments, be processed from a pre-crRNA sequence. In an embodiment, the guide RNA sequence may include natural or synthetic nucleic acids, for example modified nucleic acids such as, without limitation, locked nucleic acids (LNA), 2′-o-methylated bases, or even ssDNA (single stranded DNA). In many embodiments, the disclosed Cas proteins are selected from the Cas12 group, which may be derived from various sources known to those of skill in the art.

“Coding sequences” are DNA sequences that encode polypeptide sequences or RNA sequences, for example guide RNAs. Coding sequences that encode polypeptides are first transcribed into RNA, which, in-turn, may encode the amino acid sequence of the polypeptide. Some RNA sequences, such as guide RNAs may not encode amino acid sequences.

“Native,” “naturally-occurring,” “unmodified” or “wild-type” describe, among other things, proteins, amino acids, cells, nucleobases, nucleic acids, polynucleotides, and organisms as found in nature. For example, a nucleic acid sequence that is identical to that found in nature, and that has not been modified by man is a native sequence.

“Recombinant,” “engineered,” and “modified” as used herein, means that a particular nucleic acid (DNA or RNA) is the product of human intervention, and is not generally found “in nature.” Specifically, the particular sequence has been isolated and/or modified by one or more of in-vitro synthesis, mutation, deletion, substitution, cloning, cleavage, ligation, and amplification.

“Label” or “labelling” refers to a component with molecule that renders the component identifiable by one or more techniques.

The following disclosure is understood not to be limited to particular embodiments described below. Moreover, the provided terminology is not meant to be limiting.

### DETAILED DESCRIPTION

Embodiments disclosed herein include devices, compositions, methods, and systems for detecting the presence or absence of specific target nucleic acid sequence (e.g. double-stranded DNA sequences) in a sample, using a solution-based assay. In an embodiment, the devices, compositions, methods, and systems may be useful in rapid, sensitive, and cost-effectively identifying a patient or sample having a viral, bacterial, parasitic, or fungal infection, or a condition, disease, or disorder that may be identified by the presence of one or more specific nucleic acid sequences. In an embodiment, the disclosed methods may be practiced with simple, relatively inexpensive equipment that may be readily available. In an embodiment, the disclosed devices, compositions, methods, and systems may be useful in genetic screening, cancer screening, mutational analysis, single nucleotide polymorphism analysis, etc.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Clustered regulatory interspaced short palindromic repeats (CRISPR) were discovered in the late 1980s. While the notion that these sequences are involved in bacterial defense systems was suggested over the subsequent decades, it was not until the mid to late 2000s that it became more widely accepted. During that time several papers elucidated the basics of this acquired immunity system: foreign DNA sequences (e.g. from plasmids and viruses) flanked by palindromic repeats are incorporated into the host genome, and their RNA products direct Cas complexes to cut nucleic acids containing complementary sequences.

Simplified complexes of CRISPR-associated (Cas) proteins in combination with engineered guide RNAs were shown to be able to locate and cleave specific DNA sequences. This led to an explosion of novel technologies, especially genome editing. Further research has shown that these proteins may be used to edit genomes in vivo. CRISPR systems are found in archaea and a number of bacteria. In addition to their more widely recognized ability to target DNA, some types of Cas proteins also have indiscriminant nuclease activity. For example, the Cas12a protein has single-stranded and double-stranded endonuclease activity, and Cas13a protein has a single stranded RNase activity.

Endonuclease (DNase) enzymes cleave polymeric deoxyribonucleic acids (DNA) to produce nucleotides or shorter polynucleotides. Most DNases are known to target double-stranded DNA, while some DNases target single-stranded DNA. Cas12a belongs to the type V CRISPR system, and possesses single-stranded and double-stranded DNase activity. Specifically, Cas12a is activated when it binds to a target-DNA (or activator-DNA) using its guide RNA. In an embodiment, the target or activator DNA may be a complementary single-stranded sequence or double-stranded sequence.

Cas12a (Cpf1) is therefore a CRISPR DNase with both sequence specific and non-specific nuclease activity. Like other Cas proteins, Cas12a is “programmed” upon binding a guide RNA sequence. Cas12a is activated by binding either single-stranded or double-stranded DNA, and has the ability to cleave double-stranded DNA at targeted and non-targeted sequences. Cas12 also possesses indiscriminate single-stranded DNase activity. The enzymatic activity associated with non-specific (indiscriminant) DNase activity appears to be much higher than the targeted activity.

Ribonuclease (RNase) enzymes cleave polymeric ribonucleic acids (RNA) to produce nucleotides or shorter polynucleotides. Although some RNases are known to target double stranded RNA, most RNases target single-stranded RNA.

Cas13a belongs to the type VI CRISPR system, and possesses single-stranded RNase activity. Specifically, Cas13a is activated when it binds to a complementary target-RNA (or activator-RNA) using its guide RNA. For the single-subunit Cas13a protein, activation unlocks dual RNase functions (1) processing or maturation of a precursor crRNA (pre-crRNA) to a mature crRNA upon guide binding and (2) RNA-activated non-specific single-stranded RNase activity. The RNA-activated non-specific ssRNase activity leads to cleavage of the activator-RNA (cis-cleavage) and collateral cleavage activity on other RNA substrates (trans-cleavage). Trans-cleavage is thought to trigger bacterial programmed cell death in an effort to prevent viral replication and dissemination. As is well known in the art, single-stranded RNA sequences may include structured assemblies such as stem loops and bubbles. Thus, for example, target sequences may include partially structured assemblies.

Like several other CRISPR proteins, Cas12a's and Cas13a's guide RNA sequence may be modified, or “programmed,” to recognize and bind specific target nucleic acid sequences. This type of “programming” renders the protein's collateral cleavage function specific for a given target nucleic acid sequence. In nature, this may be a foreign or viral sequence, but when engineered in-vitro, the target sequence can be selected from any single or double-stranded sequence complementary to the engineered portion of the guide RNA.

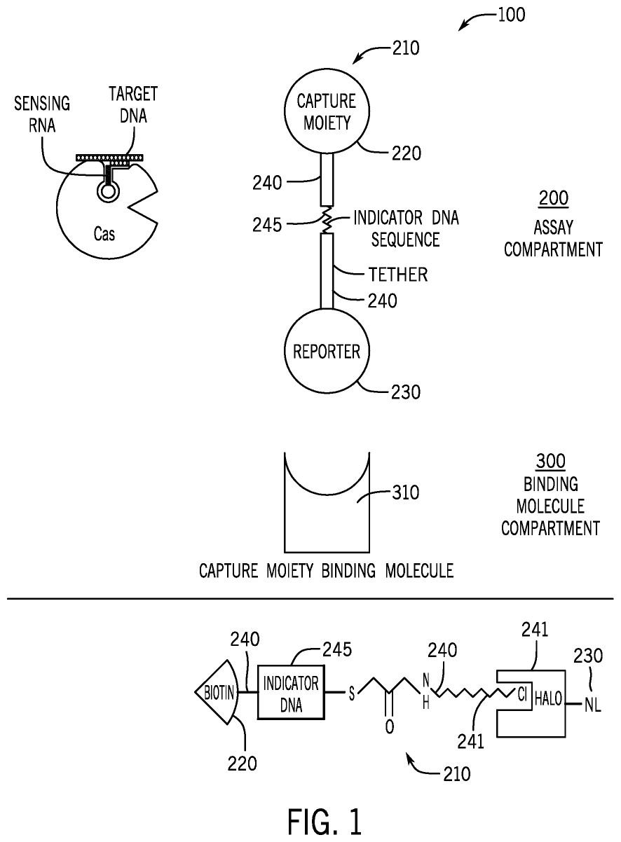

Embodiments disclosed herein include devices, compositions, methods, and systems for detecting the presence or absence of specific single-stranded or double-stranded target nucleic acid sequences, depending upon the type of Cas protein used. In some embodiments, such as that depicted in FIG. 1, the disclosed device 100 includes an indicator device 210 that further includes a soluble reporter molecule 230 connected to a capture moiety 220 via a tether molecule 240 having at least one indicator sequence 245.

The indicator device 210 is designed to react with a Cas protein in solution. In an embodiment, the disclosed device may further comprise an assay compartment 200 wherein the indicator device 100 may interact with the Cas protein. In an embodiment, an activated Cas protein (i.e. one that has bound a target nucleic acid sequence) may cleave the indicator sequence 245, and untether the reporter 230 from the capture moiety 220. An untethered reporter 230 may be separated from a capture moiety 220 (untethered and tethered) by introducing a capture moiety binding molecule 310 to the solution that binds the capture moiety 230. Binding the capture moiety 220 to the capture moiety binding molecule 310 may aid in removing and/or sequestering tethered reporter 230 molecules. Removal and/or sequestering of untethered reporter molecules 230 changes the concentration of reporter molecules in solution, thereby indicating the presence (slightly changed or unchanged concentration) or absence (reduced concentration) of the target nucleic acid sequence. In some embodiments, the device 100, may include a capture moiety compartment 300 to where the capture moiety binding molecule is removed to or sequestered.

In some embodiments, the assay compartment 200 may comprise multiple compartments, which may aid in amplifying a signal from the disclosed devices and methods. For example, the assay compartment 200 may comprise two distinct compartments to house two different reactions. The first compartment may include a tethered first molecule, such as an enzyme, (for example a protease, a restriction enzyme, a nuclease, DNase, RNase H, ribozyme, deoxyribozyme, etc.), or the like. Upon cleavage of the tether, the first molecule may be released. The first molecule may migrate or be transferred to the second compartment. The second compartment may include a second tethered molecule, such as a detection molecule (e.g., the indicator device 210). The first molecule may cleave the tethered second molecule, releasing the molecule to be detected. The steps described above with respect to the indicator device 210 would apply to this second molecule.

In one example, the first compartment includes a ssDNA molecule (e.g., the first molecule) and the second compartment includes various DNA strands. In this example, the ssDNA is released in the first compartment and transfers to the second compartment, where it can trigger a polymerization reaction with the various DNA strands to build a branched DNA assay.

The lower portion of FIG. 1 shows an embodiment of an indicator device 210 that includes a reporter 230, tether sequence 240, and capture moiety 220. In this embodiment, the reporter 230 is luciferase (here NanoLuc® (Promega), designated “NL”), which is fused to a HaloTag® (Promega, HT). The HaloTag protein is covalently bound to a chlorinated tag ligand, which in turn is covalently attached, via a succinimide group to the indicator sequence 245 at a proximal end. At the distal end of the indicator sequence, is a covalently attached capture moiety 220. In this embodiment, the capture moiety 220 is a biotin molecule. In an embodiment, the HT and chlorinated tag may be referred to as anchor molecules 241.

Kits useful for detecting a target nucleic acid in a sample are also disclosed, wherein the kit includes at least one Cas protein (also shown in FIG. 1). The Cas protein may also include a guide RNA sequence, and in another embodiment a guide RNA sequence+an activator nucleic acid sequence. In an embodiment, the disclosed kits may include indicator devices including reporter molecules tethered to capture moieties, the kit may further include one or more capture moiety binding molecule for capturing or binding to the capture moiety.

Disclosed herein are systems for the inexpensive and rapid detection of nucleic acid target sequences from a variety of sources including, without limitation mammals, viruses, bacteria, fungi, etc. In an embodiment, the system may include a Cas protein, a guide RNA sequence, an indicator device, an assay compartment, and a capture moiety binding molecule.

Methods useful in solution-based detection of target nucleic acids in a sample are disclosed. In an embodiment, the method includes combining a Cas protein, an indicator device, and a sample in a solution. In an embodiment, the sample may first be combined with a Cas protein, to form an assay mixture, and then combined with the indicator device to create a detection solution. In an embodiment, the order of addition of these three components (sample, indicator device, and Cas protein) may vary. The method includes incubating the Cas protein with the indictor device, in solution, for a time sufficient to allow cleavage of the tether sequence if the Cas protein is activated. The solution is then exposed to a capture moiety biding molecule for a time sufficient to allow the capture moiety to bind the binding molecule. The binding molecule is then removed from the solution or sequestered, and the concentration of reporter molecule in the solution is assayed. In some embodiments, the solution may be removed after sequestration of the binding molecule (for example by centrifugation or magnetic attraction of the binding molecule). If the concentration of reporter molecule in the solution is unchanged, slightly changed, or is high compared to a control solution (lacking the sample) the sample is scored as being positive for the presence of the target nucleic acid sequence in the sample. Alternatively, if the concentration of reporter molecule in the solution is very low, or lower than the control solution, the sample is scored as being negative for the presence of the target nucleic acid sequence. In some embodiments, the method may be helpful in determining the amount of target nucleic acid sequence in the sample, rather than simply a binary, ‘yes’ or ‘no,’ result.

The disclosed devices, systems, and methods provide for minimal sample preparation, while quickly and accurately determining the presence (and in some cases the amount) of target nucleic acid in the sample. The samples may be biological samples from a human or non-human patient, or an environmental sample from water, food, etc. In some embodiments, the disclosed solutions, devices, methods, and systems may include a soluble reporter molecule such as a fluorophore, or an enzyme, for example a phosphatase or luciferase (as described above). In some embodiments, the luciferase may be NanoLuc® (Promega) and the phosphatase may be alkaline phosphatase. This may allow for methods of detecting target nucleic acid sequences using fluorometric, luminescent, and/or colorimetric assays. In other embodiments, the reporter molecule may be a nanoparticle, for example a metal nanoparticle such as a gold nanoparticle.

### Detection Methods

Disclosed herein are detection methods that are useful in separating tethered reporter molecules from untethered reporter molecules in a solution, without hindering Cas protein activity by steric hindrance. In some embodiments, a solution containing Cas protein and indicator devices may be combined with a binding molecule having affinity for the capture moiety. In these embodiments, the binding molecule may be removed from the solution, sequestered or confined to a portion of the assay area (for example the binding molecule compartment), before assaying the solution (or a portion thereof) for reporter activity. In other embodiments the solution may be passed over a substrate having a binding molecule attached to the substrate, and then the solution assayed for the presence of untethered reporter. In some embodiments, the solution may also be assayed for reporter before exposure to the binding molecule and this result is then compared to the amount of reporter after exposure. In some embodiments, the amount of reporter bound to the binding molecule may also be assayed.

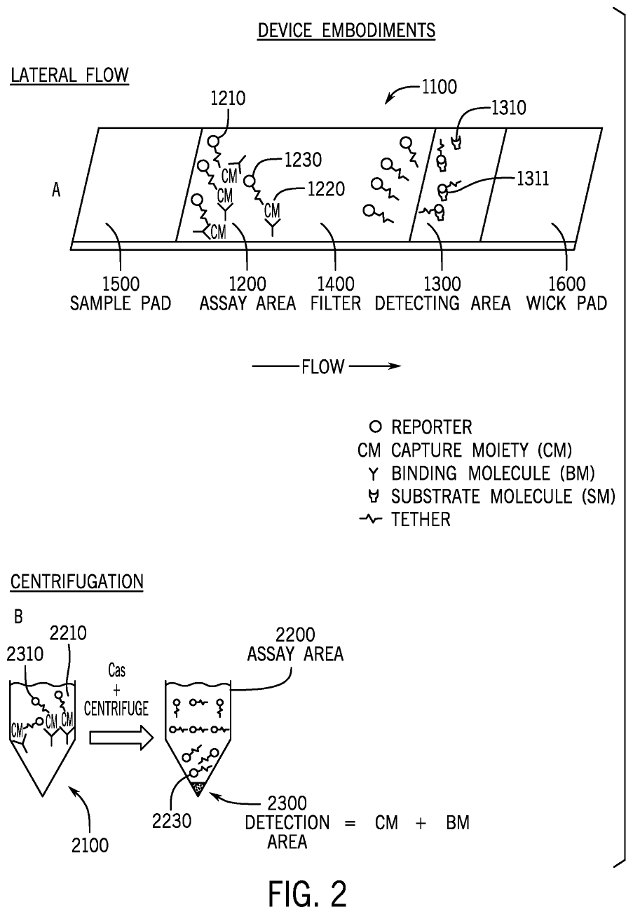

As shown in FIG. 2, detection devices useful for identifying the presence of a target DNA may include lateral flow 1100 (Panel A) or a microfuge tube 2100 (Panel B).

The lateral flow-based device 1100 may include a sample pad 1500, a capture area 1200, a detection area 1300, and a wick pad 1600 for helping to draw fluid from the sample pad 1500. In these embodiments, the reporter molecule 1230 may bind to a binding molecule at the capture area 1200. Untethered reporter then may flow through the filter to a detecting area, which may have a support structure for binding the reporter molecule.

Solution containing Cas protein, indicator devices, and sample may be applied to the sample pad. In other embodiments, the device and/or Cas protein may be located at the sample pad prior to addition of the sample. The solution (and soluble reporter, indicator device, etc.) travels away from the sample pad through the capture area and into the detecting area. In some embodiments, the presence of reporter at the detecting area is assayed with techniques other than capture by a substrate molecule. In other embodiments, a capture moiety binding molecule may be added to the solution before it is added to the sample pad, and the binding molecule may be captured at the capture area. Alternatively, the binding molecule may be sufficiently large such that it cannot traverse the filter 1400, preventing passage of tethered reporter through the filter 1400 to the detection area. In some embodiments, the binding molecule may be magnetic and the filter and/or capture area may be oppositely magnetic, such that the binding molecule is attracted to and trapped by the filter surface.

The microfuge tube-based embodiment shown in the lower portion of FIG. 2 (Panel B) may be used with methods that separate tethered from untethered reporter molecules in the solution. In this embodiment, tethered reporter molecules 2310 may be sequestered in a portion of the assay area, or may remain in the microfuge tube as solution containing untethered reporter is removed. In some embodiments, centrifugation may force tethered reporter molecules, bound to the binding molecule, to the bottom of the tube. In this embodiment, the amount or concentration of reporter remaining in the solution can be assayed, or the amount of reporter bound to the binding molecule can be assayed, or both. In an embodiment, the binding molecule may be magnetic and may be attracted to a magnetic surface near or within the microfuge tube.

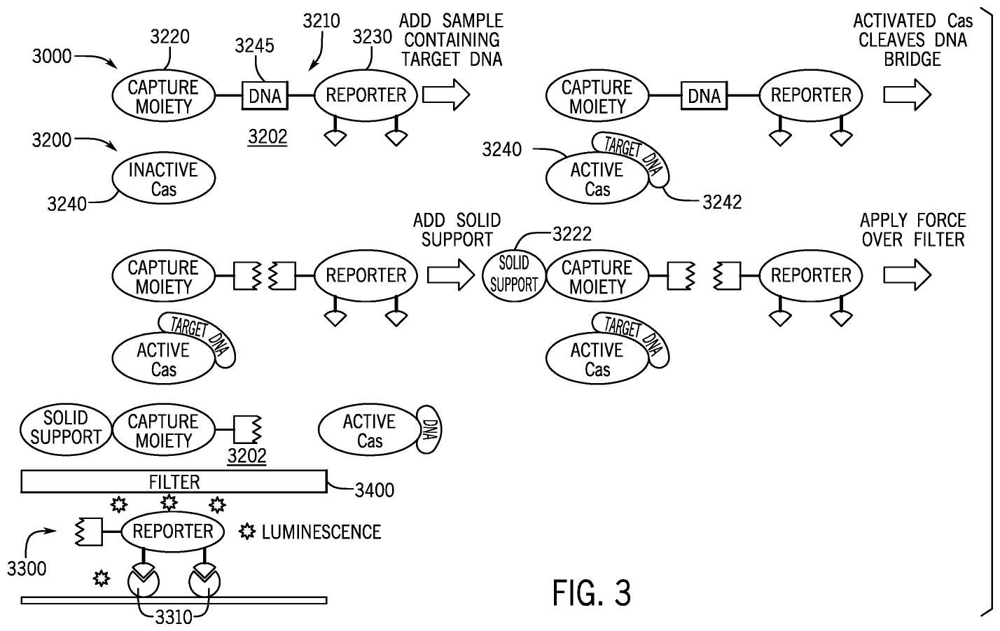

Other embodiments may include microfluidic devices, and/or may combine aspects of the devices shown in FIG. 2. For example, shown in FIG. 3 is a device 3000 with an assay area 3200, and a detection area 3300. The assay area 3200 holds a solution 3202 including an indicator device 3210 and an inactive Cas protein 3240 and the detection area 3300 includes a capture molecule 3310. The indicator device 3210 includes a reporter molecule 3230 tethered to a capture moiety 3220 (e.g., biotin or streptavidin) by an indicator sequence 3245. A biological sample containing target sequence may be added to the assay solution 3202. The presence of target sequence 3242 activates Cas protein 3240. Activated Cas protein cleaves the indicator sequence 3245, untethering the reporter 3230 from the capture moiety 3220 in the solution 3202. A binding molecule 3222 (e.g., a bead) may be added to the solution 3202. As shown, the binding molecule 3222 couples with the capture moiety 3220. A force may be applied, such that the solution including the untethered reporter molecule 3230 to the detection area 3300, while the binding molecule 3222 coupled to the capture moiety 3220 is trapped, removed, or sequestered.

In one example, the solution containing the binding molecule-capture moiety conjugates and untethered reporter molecules is flowed over a surface that reacts with either or both of the binding molecule and the capture moiety. Any tethered reporter molecules are captured by the surface. Any untethered reporter molecules can be measured in the flow through solution. A reporter molecule 3230 that passes through to the capture molecule 3310 in the detection area 3300 producing a detectable signal. For example, the signal may be luminescence. By measuring the reporter in the flow-through solution, the presence of target nucleic acid sequences in the biological sample can be determined. Adding the binding molecule 3222 after the reporter 3230 is untethered may reduce interference in the interaction between the Cas protein 3240 and the indicator sequence 3245. Without interference by the binding molecule 3222, there indicator sequence 3245 is more accessible to the Cas protein 3240, resulting in increased cleavage kinetics. FIG. 12 shows the increased reaction kinetics associated with adding a binding molecule after (as opposed to before) cleavage of the nucleic acid tether is cleaved. Cleaving a nucleic acid tether between a reporter molecule and a small capture moiety showed almost a 10-fold improvement in limit of detection when compared to cleaving a nucleic acid tether between a reporter molecule and a larger binding molecule.

### Device for Detecting a Single- or Double-Stranded Target Sequence

Embodiments disclosed herein include indicator devices for the detection of target nucleic acid sequences. In some embodiments, the disclosed device will include a reporter molecule, a capture moiety, and a tether molecule positioned between the indicator sequence and the reporter molecule and/or the capture moiety. The tether molecule connects, directly or indirectly, the reporter molecule to the indicator sequence and/or the capture moiety to the indicator sequence.

The indicator device includes at least one indicator sequence. The indicator sequence may be susceptible to cleavage by a nuclease. In an embodiment, the nuclease is a Cas protein, for example an activated Cas12a DNase or Cas13a RNase. The indicator sequence may be single-stranded RNA, double-stranded RNA, double-stranded DNA, or single-stranded DNA. The indicator sequence may be between 2 nucleotides and 50 nucleotides, or more, in length. The tether may include two or more indicator sequences of the same or different sequence. In some embodiments, the indicator sequence may be connected to a non-nucleic acid molecule at its 5′ and/or 3′ ends, or positioned between two or more indicator sequences. In some embodiments, the indicator sequence may include a thiol or biotin at the 5′ and/or 3′ end.

Tether molecules may be one or more of single-stranded RNA, single-stranded DNA, double-stranded DNA, ribonucleotides, deoxyribonucleotides, lipids, peptides, carbohydrates, polyethylene glycol (PEG), “click” chemistry tags, biotin, streptavidin, DNA, maleimide, sulfur, thiol, amino acids, proteins, peptides, primary amines, succinimide, bacterial proteins, synthetic proteins, haloalkane dehalogenase (HaloTag), chloroalkane, triazol, sulfone, heterocyclic or carbocyclic small molecules, aliphatic or heteroaliphatic small molecules, inorganic species, organometallic species, radioactive molecules and combinations thereof.

The tether molecule may include at least one anchor domain, sequence, residue, or structure at a first end and/or at a second end. The anchor domains may aid in contacting and attaching the tether to the reporter molecule (reporter anchor) or capture moiety (e.g., biotin). In some embodiments, the anchor may be covalently or non-covalently bonded to the tether, reporter molecule, and/or capture moiety. In the embodiments having a non-covalent bond, the bond may be sufficiently strong to reduce disassociation in most physiologic environments. In other embodiments, the tether may be covalently attached directly to the reporter molecule and/or the capture moiety. FIG. 1 depicts an anchor structure such as HaloTag ligand.

### Reporter Molecule

The reporter molecule may be tethered to a capture moiety by the indicator sequence. In an embodiment, the reporter molecule may be easily detected when separated from the capture moiety. In some embodiments, the reporter molecule is selected from one or more of a protease, peptidase, lipase, glycase, nuclease, endonuclease, restriction endonuclease, Cas protein, fluorophore, fluorescent molecule, luminescent molecule, a protein, a fusion protein, an enzyme, a heterocyclic or carbocyclic small molecule, an aliphatic or heteroaliphatic small molecule, an inorganic species, organometallic species, radioactive molecule, ribozyme, deoxyribozyme, and combinations thereof. In some embodiments, two or more reporter molecules of the same or different type are connected to a tether or capture moiety. In some preferred embodiments, the reporter molecule may be one or more of a fluorophore, NanoLuc, firefly luciferase, Renilla reniformis luciferase, alkaline phosphatase, horseradish peroxidase, beta galactosidase, glucose oxidase, α- or β-amylase, fluorescent protein, green fluorescent protein, yellow fluorescent protein, beta-glucuronidase, fluorescein dyes, Alexa Fluors®, quantum dots, quantum nanodots, metal, and gold. In some embodiments, wherein the reporter molecule produces, directly or indirectly, a signal, the signal is one or more of luminescence, fluorescence, plasmonic resonance, turbidity, absorbance, or electrochemical.

The reporter molecule may be detected directly or indirectly by various methods. For example, where the reporter molecule is a fluorophore, for example Alexa Fluor 488, the untethered fluorophore may be detected by exciting it at a given wavelength of light and detecting an emission wavelength. In an embodiment, the untethered fluorophore signal may be compared to the signal produced by the fluorophore remaining tethered, or the untethered signal may be compared with a standard. Where the reporter is an enzyme, for example luciferase, the presence of the untethered enzyme may be detected by interaction with a substrate, such as luciferin. In these embodiments, the tethered reporter may be removed before assaying for the untethered reporter. In some embodiments, wherein the reporter molecule is a detectable molecule or enzyme, the enzyme substrate may be located away from or distal to the binding molecule, such that the enzyme will not contact the substrate unless it is not bound by the binding molecule. In some embodiments, the reporter may interact with a molecule that has affinity for, captures, recognizes, and/or binds to the reporter. For example, the reporter molecule may be a particle/compound/molecule that, when untethered from the capture moiety may be translocated to a site away from the capture moiety and be captured, for example by an antibody, binding protein, or magnetic structure designed to interact with the reporter molecule.

In an embodiment, the reporter molecule untethered from the capture moiety may be attracted to or captured by another molecule. In some embodiments, an untethered reporter may be concentrated to help enhance detection. As described above, in some embodiments, multiple, sequential reactions may help to enhance detection.

### Capture Moiety

The capture moiety is a small, soluble molecule. In an embodiment, the capture moiety may be a compound, peptide, nucleic acid, or combinations thereof. In an embodiment, the capture moiety may bind with high affinity to a capture device. In an embodiment, high affinity may refer to a dissociation constant (Kd) of greater than about 1×10⁻¹⁰ mol/L.

The capture moiety may include of one or more of single-stranded RNA, single-stranded DNA, double-stranded DNA, ribonucleotides, deoxyribonucleotides, lipids, peptides, carbohydrates, polyethylene glycol (PEG), “click” chemistry tags, biotin, streptavidin, DNA, maleimide, sulfur, thiol, amino acids, proteins, peptides, succinimide, bacterial proteins, synthetic proteins, haloalkane dehalogenase (HaloTag), chloroalkane, triazol, sulfone, heterocyclic or carbocyclic small molecules, aliphatic or heteroaliphatic small molecules, inorganic species, organometallic species, radioactive molecules and combinations thereof. In an embodiment, the capture moiety is biotin. In an embodiment, a single capture moiety may attach to a single tether.

### Cas Protein

The disclosed Cas proteins may be derived from various sources including archaea and bacteria. In some embodiments, a native Cas protein may be derived from Paludibacter, Carnobacterium, Listeria, Herbinix, Rhodobacter, Leptotrichia, Lachnospiraceae, Eubacterium, or Clostridium. In some embodiments, the native Cas protein may be derived from Paludibacter propionicigenes, Carnobacterium gallinarum, Listeria seeligeri, Listeria newyorkensis, Herbinix hemicellulosilytica, Rhodobacter capsulatus, Leptotrichia wadei, Leptotrichia buccalis, Leptotrichia shahii, Lachnospiraceae bacterium NK4A179, Lachnospiraceae bacterium MA2020, Eubacterium rectale, Lachnospiraceae bacterium NK4A144, and Clostridium aminophilum.

The presently disclosed Cas protein is homologous to a native Cas protein. In some embodiments, the disclosed Cas protein is greater than 75%, 80%, 85%, 90%, 95%, 97%, 98%, or 99%, and less than about 100%, 99%, 98%, 97%, 95%, 90%, 85%, 80%, or 75% identical to a native Cas protein sequence.

Activation of a Cas protein may include contacting one or more target sequences with a guide RNA sequence associated with the Cas protein. In some embodiments, the guide RNA of the Cas protein may help to activate the Cas protein's nuclease activity by hybridizing to a complementary single- or double-stranded target sequence.

The disclosed Cas proteins may be Cas12a or Cas13a proteins. In an embodiment, the Cas protein is a modified Cas12a or Cas13a protein that is modified, or engineered or mutated, to alter its interaction with guide or target sequences and/or to alter its nuclease activity, for example specificity, turn-over, nucleotide preferences, etc. In other embodiments, the Cas protein may be fused to another protein, peptide, or marker to aid in isolation, identification, separation, nuclease activity, target sequence binding, etc.

### Guide RNA Sequence

Guide RNAs include at least one sequence complementary to a target sequence. In some embodiments, this target-complementary sequence may be referred to as a spacer sequence, additional sequences may be referred to as scaffold sequences. In some embodiments, the spacer sequence is derived from a human (e.g. genomic DNA or transcribed RNA) or non-human source (for example a pathogen). In some embodiments, the pathogen selected may be from bacteria, viruses, fungi, and parasites. In some embodiments the pathogen may be a bacterium selected from Mycobacterium, Streptococcus, Pseudomonas, Shigella, Campylobacter, Salmonella, Clostridium, Corynebacterium, and Treponema. In some embodiments the virus may be selected from DNA or RNA viruses including Adenoviridae, Picornaviridae, Herpesviridae, Hepadnaviridae, Flaviviridae, Retroviridae, Orthomyxoviridae, Paramyxoviridae, Papovaviridae, Polyomavirus, Rhabdoviridae, and Togaviridae. In some embodiments, pathogenic fungi include Candida, Aspergillus, Cryptococcus, Histoplasma, Pneumosystis, and Stachybotrys.

In other embodiments, the spacer RNA sequence is complementary to a non-pathogen. For example, the spacer RNA sequence may be engineered to hybridize to any nucleic acid sequence of interest. In some embodiments, the guide RNA sequence may be engineered to be complementary to a mammalian sequence of interest, for example a genomic sequence, or transcribed sequence (mRNA, microRNA, etc.). In various embodiments, the guide RNA may include a sequence complementary to a sequence associated with a mammalian condition, disease, or disorder, such as cancer, viral infection, bacterial infection, fungal infection. In some embodiments, the guide RNAs may be complementary to an mRNA or micro RNA, for example a microRNA sequence in a microRNA signature. In some embodiments, the guide RNA sequence may be within a precursor RNA, which may, in turn be part of an array with a plurality of guide RNA sequences. In some embodiments, precursor RNA sequences may be processed by the Cas protein to provide guide RNA sequences.

Guide RNA sequences include the spacer sequence, which is complementary to the target sequence, and a more constant sequence that is 5′ of the spacer sequence. This constant sequence may be referred to as a scaffold sequence, repeat, handle, or constant region and aids in binding the guide RNA to the Cas protein. In some embodiments, the constant sequence can be replaced with that of an evolutionarily related constant sequence. As is known in the art, Cas proteins may be grouped into different families comprising functional groups that recognize orthogonal sets of crRNAs and possess different nucleotide cleavage specificity. In some embodiments, the constant sequence can be modified to improve affinity and stability by including naturally occurring and synthetic or non-natural nucleobases or backbone modifications. In some embodiments, the constant sequence may include a precursor sequence. In an embodiment, a pre-crRNA sequence may be processed to form a crRNA sequence, which includes the guide sequence.

A Cas protein comprising a guide RNA may be referred to as a “programed” Cas protein. Guide RNA sequences may be introduced to and bound by a Cas protein. For example, the guide RNA may contact the Cas protein in a cell or outside a cell. Various methods may be used to contact the guide RNA with the Cas protein to produce a programmed Cas protein. In some embodiments, contacting requires less than about 2 hours, for example less than about 90 min., 60 min., 40 min., 30 min., 20 min., 10 min., 5 min., 4 min., 3 min., 2 min., or 1 min.

In some embodiments, multiple guide RNAs may be used to detect a target sequence. In these embodiments, the multiple guide RNAs may be directed to multiple regions of a target sequence having two or more sequences that may be recognized by two or more guide RNAs. When multiple guide RNAs are used to detect multiple regions of a target sequence, sensitivity (e.g., limit of detection or LOD) may be increased. The more than one guide RNAs may define a pool that is said to be diverse, wherein the two or more guide RNAs may be complementary to two or more different sequences in a given target sequence. The use of multiple guide RNA sequences complementary to multiple regions or sequences within a target sequence may help to increase the likelihood of detecting a given target sequence. This may, in turn, provide for a lower limit of detection of the disclosed method.

Multiple different guide RNAs may target multiple different target sequences. The different target sequences may overlap or may be separated by 1 or more nucleotides. In some embodiments, different guide sequences may recognize complementary sequences separated by between 10 and 200 nucleotides, or more. In many embodiments, the number of different guide RNAs may be greater than 2, for example, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more different guide RNAs may target a sequence, gene, or genome of a given organism (e.g. plants, mammals, parasites, amoebae, viruses, bacteria, fungi, etc.). In some embodiments, guide sequences may recognize between about 45 and 3,000 bp of target sequence, for example 1,300 bp. In some embodiments, guide RNAs may, together target sequences greater than 3,000 bp of a given organism. When different guide RNAs are combined, the mixture may be referred to as a pool, and its diversity may be described as a ratio of guide RNAs to Cas protein.

In one example, soluble substrate assays may have a 12:1 ratio of guide RNA to Cas protein—that is, the diversity of guide RNAs is 12. This may improve the LOD by at least four orders of magnitude from a 1:1 ratio of guide RNA to Cas protein. The LOD may vary between a pM range (e.g., for a 1:1 ratio of guide RNA to Cas protein), an fM range (e.g., for a 4:1 or 6:1 ratio of guide RNA to Cas protein), or an aM range (e.g., for a 12:1 ratio of guide RNA to Cas protein). The higher ratio of guide RNA to Cas protein is particularly useful with larger strands of target sequence. For example, a target DNA sequence may have a length that is between about 24 base pairs to about 2000 base pairs. As one example, the target DNA sequence may be 45 base pairs long (e.g., a short strand) or 1300 base pairs long (e.g., a long strand). To improve the LOD for a longer base pair sequence, a larger pool of guide RNA may be used.

### Target/Activator Sequences

Target nucleic acid sequences may be identified from various sources, including, without limitation, plants, mammals, parasites, amoebae, viruses, bacteria, and fungi. In some embodiments, the target or activator sequence is a microbial or viral sequence, in still other embodiments the target sequence is a mammalian genomic or transcribed sequence. In some embodiments, the source may be a human, non-human, or animal. In some embodiments, an animal source may be a domesticated or non-domestic animal, for example wild game. In some embodiments, the domesticated animal is a service or companion animal (e.g. a dog, cat, bird, fish, or reptile), or a domesticated farm animal.

For target sequences from pathogenic sources, the pathogen may have significant public health relevance, such as a bacteria, fungus, or protozoan, and the target sequence may be found, without limitation, in one or more of Chlamydia trachomatis, Neisseria gonorrhoeae, Trichomonas vaginalis, Ureaplasma species, Plasmodium falciparum, Plasmodium vivax, Mycobacterium ulcerans, Eschericia coli 0157:H7; Hepatitis B; human papillomavirus; influenza A, B, C, or D virus; human immunodeficiency virus; or herpesviruses.

### Capture Moiety Binding Molecule (Binding Molecule)

The binding molecule may be various structures or substances, for example at least one of a bead, particle, surface, or fiber having an affinity for the capture moiety. The binding molecule couples, binds, links, etc. to the capture moiety with high affinity. The binding molecule may be comprised of one or more of amino acids, carbohydrates, nucleic acids, proteins, peptides, glass, metal, polymer, cellulose, sephacryl, agarose, acrylamide, or dextran. In many embodiments, the binding molecule may be placed in solution, or maybe coupled or bound to a solid surface, for example glass, metal, polymer, cellulose, sephacryl, agarose, acrylamide, or dextran. In some embodiments, a plurality of capture moieties may attach to a binding molecule—that is one binding molecule may bind multiple capture moieties. In other embodiments, a single binding molecule may attach to a single capture moiety. In some preferable embodiments, the binding molecule may be a magnetic bead or a cellulose binding protein.

### Assay Area or Assay Compartment

Embodiments of the disclosed devices may include an assay area. In an embodiment, the assay area may comprise a volume for containing a solution with at least one first indicator device comprising. In an embodiment, the assay area may be configured to detect a reporter molecule in the solution. The assay area may further include a second volume that is less than the volume of the assay, for locating, confining, sequestering, concentrating or otherwise localizing a binding molecule. In an embodiment, wherein the assay area is a test-tube, the second volume may be the bottom of the test tube, or a side of the test tube, for example a side located near a magnetic substance.

### Biding Molecule Compartment or Binding Molecule Area

Embodiments of the disclosed devices may include a binding molecule compartment or area. This area or compartment may be useful for detecting the presence of a tethered reporter molecule and/or for sequestering tethered reporter. In an embodiment, the binding molecule compartment is separate from and distal to the assay area. For example, in embodiments where the device is part of a lateral flow device, the detection area may be a test line or control line. In other embodiments, as described above, the binding molecule area may be part of the assay area.

The binding molecule area may include a protein or molecule that may capture or bind the capture moiety, or may capture or bind to the binding molecule. In these embodiments, the binding molecule may aid in transporting, localizing, fixing, and/or concentrating the tethered reporter molecules. This may, in turn, allow for concentrating a signal from the untethered reporter molecule and therefore increase sensitivity of an assay for detecting the untethered reporter molecules.

### Detection Compartment or Area

A capture molecule may have affinity for the reporter molecule and be located in a detection area or compartment. In an embodiment, the capture molecule is an antibody or monobody. In these embodiments, the reporter molecule may be modified to include a tag that may be bound by the capture molecule. In other embodiments, the capture molecule may be a magnetic particle that may interact, magnetically, with the reporter molecule. In these embodiments, cleavage of the indicator device may result in a soluble reporter and a soluble capture moiety. The capture moiety may be bound by the binding molecule in a binding molecule compartment and the reporter may be bound by a capture molecule, which may be located in a detection area or compartment.

### Methods

Methods disclosed herein include methods of making the disclosed devices and methods of using same to detect a target sequence in a sample. The disclosed methods may be useful in detecting a target nucleic acid sequence in a sample, for example a biological sample, without the need for amplification of genetic material within the sample.

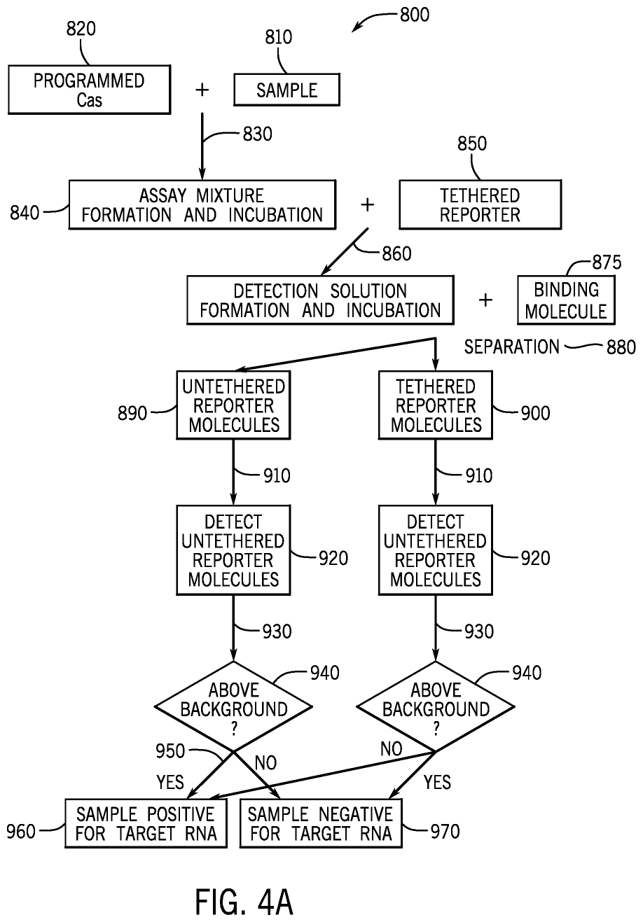

An embodiment of the disclosed method 800 is depicted as a flow diagram in FIG. 4A. In this embodiment, the Cas protein is a programmed Cas protein 820 (including a guide RNA) that is combined 830 with a sample 810 that may or may not include a target DNA sequence, and incubated to create an assay mixture 840. After incubation, the assay mixture 840 may be combined 860 with an indicator device 850 comprising a reporter molecule tethered to a capture moiety via a tether comprising at least indicator sequence, to create a detection solution 870. Other embodiments may combine the components in a different order. The detection solution 870 is incubated for a time to allow cleavage of the indicator sequence, separating the reporter from the capture moiety. Then a binding molecule 875 is added to the solution to bind the capture moiety. Tethered reporter molecules 900 coupled to the binding molecule 875 (via the capture moiety) are separated 880 from the untethered reporter molecules 890. The untethered reporter molecules 890 may be assayed for a signal 910 that can be detected 920. The detected signal 930 may be compared 940 to a background value to determine 950 whether the sample is scored as positive 960 or negative 970 for the presence of a target DNA sequence. Alternatively, the tethered reporters are assayed and may be compared to a background.

The disclosed methods are useful for assaying a variety of samples, including biological samples from a human or non-human source. In some embodiments, the samples may be selected or derived from one or more of blood, sweat, plasma, serum, sputum, saliva, mucus, cells, excrement, urine, cerebrospinal fluid (CSF), breast milk, semen, vaginal fluid, tissue, etc. Target nucleic acid sequence detectable by the disclosed methods may be derived from a variety of sources or may be synthetically produced. Where the target nucleic acid sequence is biologically derived, the source may be one or more of a fungus, bacterium, virus, protozoa, eukaryote, mammalian cell, or human cell.

The disclosed methods may use a variety of detection methods to determine the presence or absence of a target nucleic acid sequence. Detection may be direct or indirect detection of a reporter molecule untethered from the capture moiety or tethered to the capture moiety. Suitable reporter molecules may include, without limitation, one or more of a fluorescent molecule, a luminescent molecule, a fusion protein, a protein, an enzyme, a fluorescent or luminescent protein, a SERS particle, or a nanoparticle. Suitable reporter molecules may result in a signal that is detectable by one or more of Raman spectroscopy, fluorescence spectroscopy, spectroscopy, electrochemical methods, visual inspection (for example, color, turbidity), or surface Plasmon resonance. In some embodiments, the disclosed methods may include a step that may result in untethering a reporter molecule from a capture moiety. In some embodiments, the capture moiety may be biotin. In some embodiments, a plurality of tether molecules may be attached to the capture moiety, and a plurality of reporter molecules may be attached to a single tether molecule. In some embodiments, the tether molecule may include one or more of PEG, DNA, streptavidin, biotin, maleimide, sulfur, thiol, amino acids, proteins, succinimide, bacterial protein, haloalkane dehalogenase (HaloTag), chloroalkane, triazol, sulfone, and the tether molecule is covalently attached to the capture moiety and/or the reporter molecule.

### Systems

Also disclosed are systems for determining a presence of a target nucleic acid sequence. An embodiment of the disclosed system may include a modified Cas molecule, a device including a tether, a reporter molecule, and a capture moiety, wherein the tether has at least one indicator nucleic acid sequence, the Cas protein is “programmed” and includes a guide nucleic acid sequence complementary to the target nucleic acid sequence. The system may further include an assay compartment, a detection compartment, and a filter positioned between the assay compartment and the detection compartment, wherein the filter is permeable to an untethered reporter molecule. In many embodiments, the system may further include at least one detector configured to detect a signal from an untethered reporter within the detection area.

In some embodiments, the system may include a digital computer as shown in FIG. 4B. The system 5000 may include a detection device 5100, and at least one detector 5200 configured to detect a signal from an untethered reporter within a detection area of the detection device 5100. The system may further include an input device 5300, an output device 5400, a storage device 5500, a memory unit 5600, and a digital computer 5700 (or central processing unit, CPU), all of which may be in electrical communication with a bus 5800. The detector and detection device may be in direct communication, for example light or electrical communication. The CPU may include processing electrical circuitry configured for accepting a signal from the detector and processing the signal. The input and/or output devices may provide for user interface, such as for monitoring the system and/or the signal. In an embodiment, the system may indicate the presence or absence of a target sequence in a given sample.

Such a digital computer is well-known in the art and may include one or more of a central processing unit, one or more of memory and/or storage, one or more input devices, one or more output devices, one or more communications interfaces, and a data bus. In some embodiments, the memory may be RAM, ROM, hard disk, optical drives, removable drives, etc. In some embodiments, storage may also be included in the disclosed system. In some embodiments, storage may resemble memory that may be remotely integrated into the system.

The disclosed system may further include at least one output device, for example one or more monitors, display units, video hardware, printers, speakers, etc. In some embodiments, at least one output device is a monitor for viewing the diagnostic video. One or more input devices may also be included, for example pointing devices (e.g., mouse), text input devices (e.g., keyboard), touch screen, cameras, detectors, etc. In some embodiments, at least one input device is a detector for receiving a signal resulting from untethered reporter molecules in the detection area. In some embodiments the detector may be a single wavelength or broad spectrum detector.

The disclosed system may further include at least one communications interface, such as LAN network adapters, WAN network adapters, wireless interfaces, Bluetooth interfaces, modems and other networking interfaces.

The disclosed system may further include one or more data buses for communication among the various parts of the disclosed system, for example input/output buses and bus controllers.

In some embodiments, the disclosed system may comprise one or more distributed computers, and may be implemented in various types of software languages including, without limitation C, C++, COBOL, Java, FORTRAN, Python, Pascal, among others. The skilled artisan may compile various software source codes into executable software for use with the disclosed system.

### Methods for Detecting a Target Nucleic Acid Sequence

Various methods for detecting a target nucleic acid sequence in a sample are disclosed. In an embodiment, the method may include (i) combining a sample with a Cas protein, wherein the Cas protein includes a guide RNA having a spacer sequence that is complementary to a target sequence of interest, to create an assay mixture, (ii) incubating the assay mixture to allow a target nucleic acid in the sample to hybridize to the spacer sequence, (iii) combining the assay mixture with the described device to create a detection solution, (iv) incubating the detection solution, (v) adding a binding molecule to the detection solution to create a separable detection solution, (vi) incubating the separable detection solution to allow the binding molecule to bind the capture moiety in the device, (vii) applying a force to the separable detection solution sufficient to separate an untethered reporter molecule from tethered reporter molecule bound, via the capture moiety in the device, by the binding molecule; and (v) detecting the untethered reporter or the tethered reporter.

The disclosed method may include combining a Cas protein with the sample, which may be a test sample that may or may not include a target nucleic acid sequence. In these embodiments, the Cas protein includes a guide RNA sequence. In some embodiments, the method includes a step of combining the Cas protein with a guide RNA sequence prior to combining it with the sample. In various embodiments, the sample that may be combined with a plurality of Cas proteins may include a plurality of guide RNAs of the same or different sequence. In these embodiments, the method may be able to detect the presence of one or more target sequences in a sample. The Cas protein is combined with the sample to create an assay mixture. As described below, the sample may be other than a test sample, for example the sample may be a control sample (known to have no target nucleic acid sequences) or a standard sample having a known amount of target sample.

Samples may be obtained from various sources, and may include various nucleic acid sequences. In some embodiments, the sample may be obtained from a biological, environmental, or synthetic source. A biological sample may include, for example, a tissue, cell, or a bodily fluid from a subject, as well as lysates thereof. In many of these embodiments, the subject may be human, non-human, eukaryotic, prokaryotic, etc. Samples may also be obtained from agricultural and veterinary subjects, including plants and animals. Samples that are environmental may be obtained from foodstuffs, industrial, commercial, medical, and environmental surfaces, air samples, and water samples. In some embodiments, bodily fluid may include blood, sputum, serum, plasma, urine, sweat, saliva, mucus, cells, organelles, etc.

A programmed Cas protein may be combined with the sample and incubated for any suitable times. In some embodiments, the sample is incubated with the Cas protein for less than about 2 hrs., 90 min., 60 min., 40 min., 30 min., 20 min., 10 min., 5 min., 4 min., 3 min., 2 min., 1 min., 55 sec., 50 sec., 40 sec., 30 sec., 20 sec., or 10 sec., and more than about 5 sec., 10 sec., 20 sec., 30 sec., 40 sec., 50 sec., 60 sec., 2 min., 3 min., 4 min., 5 min., 10 min., 20 min., 30 min., 40 min., 50 min., 60 min., or 90 min.

The assay mixture may be incubated under various conditions to allow a target nucleic acid sequence, if present in the sample, to hybridize to the spacer sequence of the guide RNA. In some embodiments, the conditions are designed to aid in hybridization of RNA-RNA, RNA-DNA, or DNA-DNA sequences (e.g. spacer RNA sequence to target nucleic acid sequence), wherein the sequences are 100% complementary. In other embodiments, the conditions for incubation of the assay mixture may be varied to allow for less than 100% complementarity between the spacer sequence and the target sequence, for example 1 mismatch between target nucleic acid and spacer RNA, or less than about 2 mismatches, 3 mismatches, 4 mismatches, or 5 mismatches. In some embodiments, hybridization between a target DNA and a guide RNA may activate non-specific DNase activity of a Cas12a protein, when complementarity is greater than about 80%.

Target sequences may be any single-stranded or double-stranded nucleic acid sequence, for example single-stranded or double-stranded DNA. The target sequence may be derived from coding or non-coding DNA, cDNA, mRNA, tRNA, rRNA, iRNA, miRNA, coding and non-coding RNA. In some embodiments, the target nucleic acid is derived from a pathogen such as a microbe, bacterium, fungus, or virus.

The assay mixture, which may or may not include an activated Cas protein, may be combined with an indicator device, as described above, to create a detection solution. In some embodiments, the detection solution is designed to allow an activated Cas protein to cleave an indicator nucleic acid sequence. The indicator device may be configured with the indicator sequence available to the Cas protein, free of interference from the reporter or capture moiety, allowing the activated Cas protein to cleave the indicator sequence. In an embodiment, the indicator sequence is selected from single-stranded and double-stranded nucleic acid. In some embodiments, an activated Cas protein will cleave an indicator sequence in the tether of the device. Incubation conditions may be selected to reduce cleavage of the indicator sequence by Cas proteins that have not been activated, and by non-Cas proteins with DNase or RNase activity.

Nuclease inhibitors may be present in the assay mixture. In some embodiments, the assay mixture may include one or more molecules that inhibit non-Cas12a-dependent DNase activity, but do not affect DNase activity by activated Cas12a proteins. For example, the inhibitor may inhibit mammalian, bacterial, or viral DNases. In some embodiments, the DNase inhibitor may be added to the sample to help preserve a target nucleic acid sequence. In these embodiments, the method may include a step of adding one or more DNA preserving compounds to the sample, for example one or more DNase inhibitors.

The detection solution may be incubated under various conditions that may aid in cleavage of the indicator sequence in the tether. In some embodiments, the incubation conditions may help to enhance the activity of an activated Cas protein, while minimizing activity of other enzymes. In some embodiments, incubation conditions may promote cleavage of all or substantially all of the tethers present in the detection mixture, to help optimize the concentration of untethered reporter molecules.

The binding molecule may be added to the detection solution to create a separable detection solution. The separable detection solution may be incubated to allow the binding molecule to couple to a capture moiety in the device. In some embodiments, the solution is incubated with the solid support for less than about 2 hrs., 90 min., 60 min., 40 min., 30 min., 20 min., 10 min., 5 min., 4 min., 3 min., 2 min., 1 min., 55 sec., 50 sec., 40 sec., 30 sec., 20 sec., or 10 sec., or more than about 5 sec., 10 sec., 20 sec., 30 sec., 40 sec., 50 sec., 60 sec., 2 min., 3 min., 4 min., 5 min., 10 min., 20 min., 30 min., 40 min., 50 min., 60 min., or 90 min.

A force may be applied to the detection solution after sufficient incubation, to aid separating tethered and untethered reporter molecules. In these embodiments, the separable detection solution may be transferred to a separation device comprising a filter or membrane (for example a lateral flow or microfluidic device), permeable to untethered reporter molecules but not to the binding molecule coupled to the capture moiety. The separating force may allow for movement or translocation of a tethered reporter molecule away from the untethered reporter, which will remain in solution unless separately removed. The separating force may also aid in translocating untethered reporter molecules by or through a filter or membrane into a detection area.

The separating force may be the result of one or more of magnetic, gravitational, fluid, gas, or other forces. In some embodiments, the force may be applied by subjecting the detection mixture to centrifugation. In these embodiments, centrifugation may provide the force necessary for capture moieties bound to binding molecules to pellet, and/or for fluid in the separable detection solution to traverse a filter or membrane. In some embodiments, untethered reporter molecules (and other suitable solutes) may flow through the filter or membrane and enter the detection area. In these embodiments, the detection area may be part of a detection device that may be a test tube, for example a microfuge tube. In other embodiments, the detection device may be a multi-well plate.

In some embodiments, the device or detection device may be a lateral flow assay device. In these embodiments, untethered reporter molecules may traverse a filter, membrane, or pad as solvent from the sample is drawn toward a wicking pad.

Detecting the untethered reporter may be achieved in various ways. In most cases, the untethered reporter molecule is translocated to a detection area for detecting. In these embodiments, the detecting area may include a substrate that may be used to detect the presence of the reporter molecule. In other embodiments, the detecting area may include one or more molecules that may aid in concentrating and/or localizing the reporter molecule. In some embodiments, the reporter molecule may, directly or indirectly, produce a signal that may be detected by various means. In some embodiment, for example wherein the reporter molecule is an enzyme, the signal may be colorimetric, fluorescent, or luminescent. In other embodiments, for example wherein the reporter molecule is a fluorophore, nanoparticle, or includes a dye molecule, the signal may be absorbance and/or emission of light at a particular wavelength. In some embodiments the signal may be detected by visual inspection, microscope, or light detector.

The presence of a target sequence may be determined if the signal in the detection area is greater than a background signal. In these embodiments, a background signal may be determined from a sample that is known to contain no target sequence, or where the target sequence has been purposely destroyed by addition of one or more nucleases or compounds. Detecting a signal above the background signal may indicate the presence of a target sequence in the sample, where detecting no signal or a signal below the background signal may indicate no target sequence is present in the sample.

### Methods for Quantification of a Target Sequence in a Sample

In some embodiments, the disclosed devices and methods may be used to detect a target nucleic acid sequence in a biological sample and provide quantitative or qualitative information regarding the abundance of the target nucleic acid sequence. In these embodiments, the signal detected from the methods described above may be compared to a background signal and/or standard signals. A standard signal may be the result of a sample containing a known amount of reporter molecule, a known amount of target sequence, or both. In an embodiment, the reporter in the standard sample may be attached to a tether, part of a tether, or may be free of any other molecules. In some embodiments, a target sequence may be included in a standard sample, for example where the reporter is an enzyme. In an embodiment, the target sequences may be the same as, or different than, the target sequence in a test sample. A test sample is a sample obtained from a subject, patient, or source wherein the presence of a target sequence is unknown.

Quantifying the amount of target nucleic acid sequence in a test sample may include a step of measuring signal produced from untethered reporter from a standard sample having a known concentration of a target nucleic acid sequence. In these embodiments, the standard sample may include the same target nucleic acid sequence being assayed in the test sample, or it may be a reference target nucleic acid sequence that is different than the target nucleic acid sequence that may or may not be present in the test sample. In these embodiments, the guide RNA sequence is complementary to the reference nucleic acid sequence. Comparing signals produced by standard samples to a test sample may aid in determining the amount of target nucleic acid sequence in a test sample. If the detected signal is greater than a background value, the target nucleic acid sequence is present in the biological sample, and the signal may then be compared to one or more standard samples to determine the quantity of target nucleic acid sequences in the test sample.

### Levels of Target Sequence for Detecting a Signal

The disclosed devices and methods are useful for specifically and sensitively detecting a target nucleic acid sequence in various samples. In some embodiments, the disclosed devices and methods may detect a target nucleic acid sequence in a sample, wherein the concentration of target nucleic acid sequence is less than about 1×10-6 M, for example less than about 100×10⁻⁹ M, 10×10⁻⁹ M, 1×10⁻⁹ M, 100×10⁻¹² M, 10×10⁻¹² M, 1×10⁻¹² M, 100×10⁻¹⁵ M, 10×10⁻¹⁵ M, 1×10⁻¹⁵ M, 100×10⁻¹⁸ M, 10×10⁻¹⁸ M, or 1×10⁻¹⁸ M. The concentration of target nucleic acid in a sample may be quantitated by measuring the amount of a signal detected in the detection area as a result of the untethered reporter molecule.

Kits for Detecting and/or Quantitating the Level of Target Sequence

The disclosed devices and methods may be used with kits, such as test kits, for detecting pathogen infection, including active infection, in a variety of samples. In some embodiments, the sample may be derived from a chronically-infected subject or individual (human or non-human). In some embodiments, the kits may help provide a level of infection by quantitation of the amount of target nucleic acid sequence in the sample. In some embodiments, the kit may include one or more of a guide RNA sequence, and a Cas protein. In some embodiments, one or more Cas proteins are provided, wherein the Cas protein is bound to a guide RNA sequence. In these embodiments, the guide RNA sequences may be the same for each different Cas protein, and therefore the kit may be able to recognize and/or differentiate between two or more target sequences. In other embodiments the kit may include one or more nucleic acids encoding for the Cas protein and guide RNA. The kit may further include a device comprising at least a reporter molecule tethered to a capture moiety, wherein the tether includes at least one indicator sequence. The kit may further include a binding molecule with affinity for the capture moiety in the device. The kit may further include a detection device having a detection area, which may include one or more substrate molecules that may interact with untethered reporter molecules.

### Method of Making a Device for Detecting a Target Sequence

Disclosed herein are methods of constructing devices for determining the presence of a target nucleic acid sequence, the method comprising: synthesizing a tether molecule having, a first end, a second end, and at least one indicator sequence positioned between the first and second end. In some embodiments, the at least one indicator sequence may be synthesized with one or more molecules at the 3′ or 5′ end, and at least two thymine bases in between. The tether may be attached to a reporter molecule at the first end of the tether molecule, and a capture moiety at the other. In some embodiments, the molecules at the 3′ and/or 5′ end may be anchor molecules for attaching directly to the reporter molecule and capture moiety, in other embodiments, additional molecules may be positioned between the tether and reporter molecule or capture moiety. In some embodiments, the step of attaching of the capture moiety or the at least one reporter molecule includes covalently attaching to the tether by one or more of a cysteine linkage or amine linkage.

### Detection Devices

The disclosed tethered reporter molecule may be included in a detection device that further includes a filter or membrane useful in separating untethered reporter molecules from tethered reporter molecules. The detection devices may further include a detection area in a detection compartment and an assay area in an assay compartment. In some embodiment, the assay area and detection area may be separated by one or more filters or membranes, wherein the filter or membrane is permeable to the untethered reporter molecule but not to a tethered reporter molecule.

### EXAMPLES

### Example 1: Specificity of Non-Soluble Assay

Experiments were performed to examine the specificity of Cas12a/guide RNA complexes to target DNA sequences. Specifically, the Cas12a/guide complex was mixed with a complementary dsDNA sequence as well as non-complementary sequences to evaluate specificity of the complex. The amount of Alexa Fluor® 488 released upon cleavage of the ssDNA soluble substrate from each Cas12a/guide/dsDNA target combination was quantified.

The following materials were prepared. EnGen Lba Cas12a (Cpf1) was obtained from NEB (cat #M6053T) and was diluted to 4 nM in assay buffer prior to the experiment. Guide RNAs CRI-41 (UAAUUUCUACUAAGUGUAGAUCGCGCCAAAGCGACAGCCAC) (SEQ ID NO: 1), CRI-45 (UAAUUUCUACUAAGUGUAGAUGCUUGGUUGGACUUGACGAU) (SEQ ID NO: 2), and CRI-47 (UAAUUUCUACUAAGUGUAGAUAGUAUUGCCUGUGUCGUCGG) (SEQ ID NO: 3), were synthesized by Synthego and diluted to 4 nM in assay buffer prior to the experiment. dsDNA targets CRI-40 (CTGCGTGGTGCTTTACGCGCCAAAGCGACAGCCACGCATCTCGTG) (SEQ ID NO: 4), CRI-44 (CGCGGTGATACGGGCAAGTATTTTGGCTTGGTTGGACTTGACGATCATCAGGT AG) (SEQ ID NO: 5), and CRI-46 (CGTGCCTACCGTTTCAGTATTGCCTGTGTCGTCGGTTCCAGAGGTGTTC GAC-CGC) (SEQ ID NO: 6) were synthesized by IDT and diluted to 4 nM in assay buffer prior to the experiment. ssDNA substrate (Biotin-TTATTTTATT-Alexa488) (SEQ ID NO: 7) was synthesized by IDT and was diluted to 400 nM in assay buffer prior to the experiment. Assay buffer composition is as follow: 10 mM Tris-HCl, pH 7.5, 10 mM MgCl₂, 1 mM TCEP, 40 μg/mL BSA, and 0.01% Igepal CA-630. MyOne SA beads from Invitrogen (cat #65002) were diluted to 0.1 mg/mL in capture buffer (1 M NaCl, 10 mM Tris 7.5, 10 mM EDTA, 0.01% Tween-20).

Assays were performed as follows. Cas12a and guide were mixed together and incubated for 10 minutes at room temperature before the addition of target. Target dsDNA was added to Cas12a and guide and the reaction was further incubated for 10 minutes at room temperature. The reaction was initiated by adding substrate and incubated at 37° C. for 1 hour. EDTA was also present in some cases as a negative control. The final concentration of the components in the assay were as follows: 1 nM Cas12a, 1 nM Guide RNA, 10 pM Target DNA, (±45 mM EDTA), 10 nM A488-DNAb soluble substrate. Magnetic beads were then added to the reaction, which was incubated at room temperature for 15 minutes before being collected on a magnet. The supernatant was collected and assayed for fluorescence at 490/525 nm. The amount of Alexa Fluor 488 released during the cleavage reaction of the substrate by the Cas12a/guide complexes was determined using a standard curve. The background activity of each Cas12a/guide complex was also measured in the absence of target.

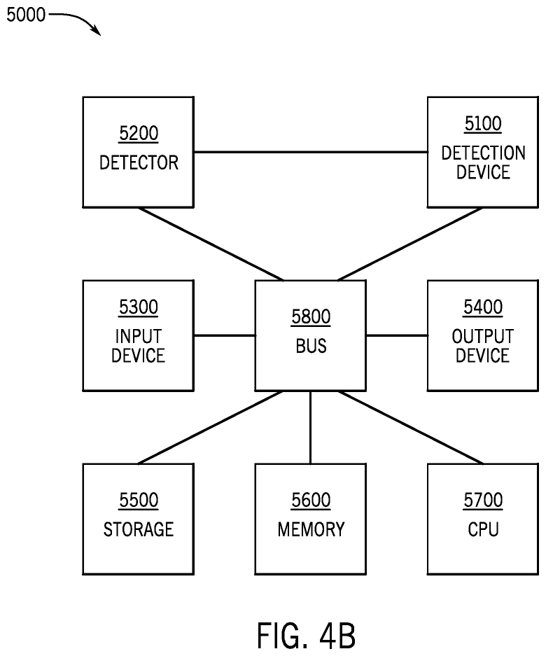

Results from these experiments are shown in FIG. 5. The results show that only the complementary pairs CRI40 and CRI41, CRI44 and CRI45, and CRI46 and CRI 47, in the absence of EDTA, are active and able to stimulate Cas12a to completely cleave the substrate, thus releasing Alexa Fluor 488. The presence of EDTA completely inhibited the reaction, as Cas12a is dependent on free magnesium ions for activity. When the Cas12a/guide complex is in the presence of non-complimentary targets, CIR41 with CRI44 or CRI46, CRI45 with CRI40 or CRI46, and CRI47 with CRI40 or CRI44, the signal is equivalent to background signal in the absence of a complimentary target. This shows that the Cas12a/guide complex activity is very specific to the presence of its complementary target.

### Example 2: Comparison of Limit of Detection (LOD) for Soluble and Non-Soluble Substrates

Experiments were performed to compare the limit of detection of a DNA target using a solution phase substrate and a substrate linked to a solid surface such as a magnetic bead. Specifically, a Cas12a/guide RNA mixture was mixed with a concentration range of target dsDNA and the amount of Alexa Fluor 488 released upon cleavage of both ssDNA substrates was quantified.

The following materials were prepared. EnGen Lba Cas12a (Cpf1) was obtained from NEB (cat #M6053T). Guide RNA CRI-41 (UAAUUUCUACUAAGUGUAGAUCGCGCCA AAGCGACAGCCAC) (SEQ ID NO: 1), was synthesized by Synthego. Cas12a and guide were mixed and diluted to 2 nM in assay buffer prior to the experiment. dsDNA target CRI-40 (CTGCGTGGTGCTTTACGCGCCAAAGCGACAGCCACGCATCTCGTG) (SEQ ID NO: 4) was synthesized by IDT and diluted to 400 pM in assay buffer prior to the experiment. ssDNA soluble substrate (Biotin-TTATTTTATT-Alexa488) (SEQ ID NO: 7) was synthesized by IDT and was diluted to 40 nM in assay buffer prior to the experiment. ssDNA substrate was linked to a magnetic bead (Bead-TTATTTTATT-Alexa488) (SEQ ID NO: 7) and diluted to 40 nM in assay buffer prior to the experiment. Assay buffer composition is as follows: 10 mM Tris-HCl, pH 7.5, 10 mM MgCl₂, 1 mM TCEP, 40 μg/mL BSA, and 0.01% Igepal CA-630. MyOne SA beads from Invitrogen (cat #65002) were diluted to 0.1 mg/mL in capture buffer (1 M NaCl, 10 mM Tris 7.5, 10 mM EDTA, 0.01% Tween-20) prior to use with soluble ssDNA substrate.

Assays were performed as follows. Cas12a and guide were mixed together and incubated for 10 minutes at room temperature before the addition of target. Target dsDNA was added to Cas12a and guide and the reaction was further incubated for 10 minutes at room temperature. The reaction was initiated by adding substrate and was then incubated at 37° C. for 1 hour. The final concentrations in the assay were as follows: 1 nM Cas12a, 1 nM Guide RNA, Target DNA at concentrations from 0 pM to 100 pM, 10 nM A488-DNA substrate. In the case of the soluble substrate, magnetic beads were then added to the reaction and incubated at room temperature for 15 minutes. For both substrates, beads were collected with a magnet and the supernatant was collected and assayed for fluorescence at 490/525 nm. The amount of Alexa Fluor 488 released during the cleavage reaction of the substrate by the Cas/guide complexes was determined using a standard curve.

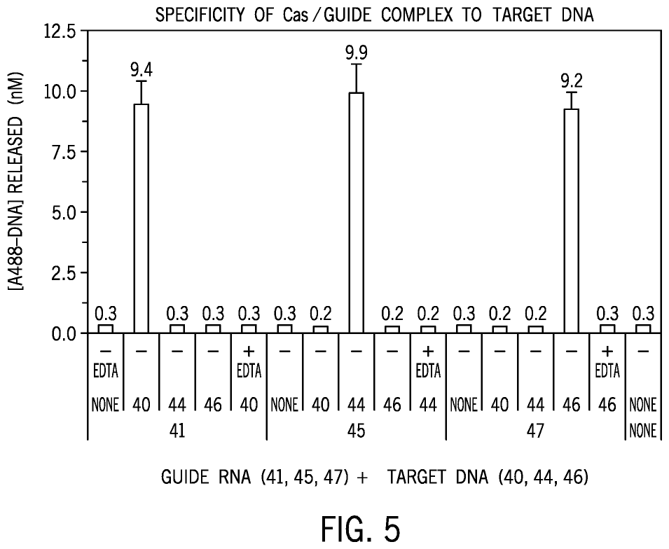

Results from the experiments are shown in FIG. 6. The calculated LOD from these curves are 0.24 pM in the case of the soluble substrate and 1.48 pM in the case of the substrate attached to a solid surface (magnetic bead). In the case of the soluble substrate, the CRISPR associated enzyme was able to cleave the indicator sequence tether more rapidly, providing a larger concentration of untethered reporter molecules in solution, which resulted in more untethered molecules creating a higher detection rate than when the reporter molecule was tethered to a solid support. This likely resulted from the CRISPR associated enzyme having wider access to the indicator sequence tether without any hindrance from the solid support.

### Example 3: Effective Cleavage of Soluble Substrate Using an Enzymatic Reporter

The use of a soluble substrate was also tested with a NanoLuc® reporter to show versatility of the assay.

The following materials were prepared. EnGen Lba Cas12a (Cpf1) was obtained from NEB (cat #M6053T). Guide RNA CRI-41 (UAAUUUCUACUAAGUGUAGAUCGCGCCA AAGCGACAGCCAC) (SEQ ID NO: 1) was synthesized by Synthego. Cas12a and guide were mixed and diluted to 2 nM in assay buffer prior to the experiment. dsDNA target CRI-40 (CTGCGTGGTGCTTTACGCGCCAAAGCGACAGCCACGCATCTCGTG) (SEQ ID NO: 4) was synthesized by IDT and diluted to 400 pM in assay buffer prior to the experiment. ssDNA soluble substrate (Biotin-CCCCCCCCCC—NH₂), synthesized by IDT and chemically linked to NanoLuc enzyme, was diluted to 4 nM in assay buffer prior to the experiment. Assay buffer composition is as follow: 10 mM Tris-HCl, pH 7.5, 10 mM MgCl₂, 1 mM TCEP, 40 μg/mL BSA, and 0.01% Igepal CA-630. MyOne SA beads from Invitrogen (cat #65002) were diluted to 0.1 mg/mL in capture buffer (1 M NaCl, 10 mM Tris 7.5, 10 mM EDTA, 0.01% Tween-20) prior to use with soluble ssDNA substrate.

Assays were performed as follows. Cas12a and guide were mixed together and incubated for 10 minutes at room temperature before the addition of target. Target dsDNA was added to Cas12a and guide and the reaction was further incubated for 10 minutes at room temperature. The reaction was initiated by adding substrate and was then incubated at 37° C. for 1 hour. The final concentrations in the assay were as follows: 1 nM Cas12a, 1 nM Guide RNA, Target DNA at concentrations from 0 pM to 100 pM, 1 nM NanoLuc-DNA substrate. At the end of the reaction, magnetic beads were added to the reaction and incubated at room temperature for 15 minutes. Beads were then collected with a magnet and the supernatant was collected and assayed for luminescence using Nano-Glo® buffer and a plate reader.

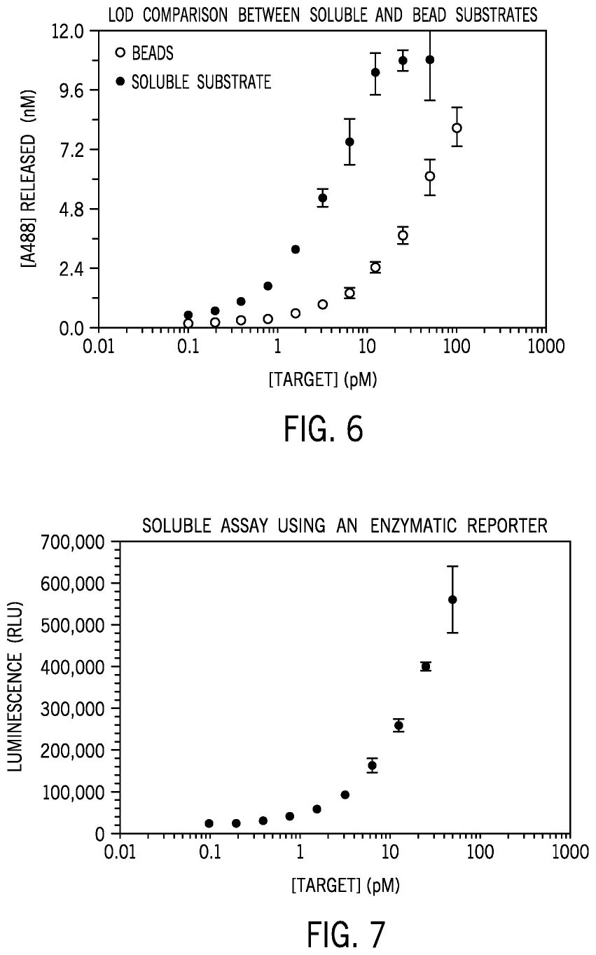

Results from these experiments are shown in FIG. 7. The soluble substrate is versatile and can be used with a variety of reporters. The limit of detection calculated from these data points is 1.1 pM.

### Example 4: Effect of Dog-Piling

Experiments were performed to look at the effect of using multiple guides (“dog-piling”) on the amount of reporter released and thus the limit of detection.

The following material were prepared. EnGen Lba Cas12a (Cpf1) was obtained from NEB (cat #M6053T). A gBlock gene fragment was synthesized by IDT (ATGATCTTTGGTGCCGCGCTATTGGGGCCGCAGTGTAGGCACGGGCTATCTGG AGAACGATCGAGTTGGTTACTGTTCGTCGTTGGTCTGGAGTTGCCCGTGCCTA CCGTTTCAGTATTGCCTGTGTCGTCGGTTCCAGAGGTGTTCGACCGCTGGAGTG CTGGTGAGCGCAGCGAGCTCATCGGATCGGAGTTTCTCGATGTGTTGGCCTCG GTGCCAGACCCGAGGGACCCACGCGGTCGGAGGTATTCGCTGATGGCTTTGTT GGCGATCGCGGTTCTGGCCACTGCCGCGGGGATGCGCGGCTATGCTGGTTTTG CCACATGGGCGGCCACCGCTTCCGATGATGTGTTGGCCCAATTAGGGGTCCGG TTCCGGCGGCCCAGTGAGAAGACCTTCCGCGCTGTTTTGTCTCGGCTAGACCC CGCCGACCTCAACGCCAGGATGGGCAGTTACTTCACTGCACACGTGGCCAGCA GCGACCCCAGTGGATTGGTGCCGATCGCGTTGGACGGCAAGATGCTGCGTGGT GCTTTACGCGCCAAAGCGACAGCCACGCATCTCGTGTCGGTGTTCGCCCACCG TGCCCGATTGGTGCTCGGTCAACTCGCTGTCGCCGAGAAAAGCAATGAAATTC CCTGCGTACGTGCCCTGCTCACGCTGCTGCCGGATAACTTGCGGTGGCTGGTC ACCGTGGATGCGATGCATACCCAGGTCGTCACCGCGAAGTTGATCTGCGCCAC CTTGAAGTCGCACTACCTGATGATCGTCAAGTCCAACCAAGCCAAAATACTTG CCCGTATCACCGCGCTGCCCTGGGCCGAGGTGCCCGCAGCCGCTACCGACGAC TCCCGCGGCCACGGCCGTGTCGAGACCCGCACCCTGCAAATCATCACCGCTGC ACGAGGAATCGGCTTCCCCTACGCAAAACAAATCATCCGGATCACTCGTGAAC GCTTGATCACCGCCACCGACCAGCGCAGCGTGGAGGTGGTCTATGCCATCTGC AGCCTGCCGTTCGAGCACGCCCGCCCTACCGCGATCATGACCTGGATGCGTCA ACACTGCGGAATCGAGAACAGCCTGCACTGGATACGCGACGTCACCTTCGAC GAAGACCGTCACAGGCCACATACCGGAAACGGCGCACAGGTCCTAGCAACGC TACGCAACACCGCGATCAATCTGCACCGCCTCAACGGCGCCGACAACATCGCC GAAGCCTGCCGGATCACCGCTTTGACCGCCAACCGCCGCCTAGACCTCCTCAA TCCACAATTCCCCAGCTCACAAGCCTGC) (SEQ ID NO: 8) and used as a target. Guide RNAs were synthesized by Synthego and used in 4 pools of five guides. Guide RNAs CRI-33 (UAAUUUCUACUAAGUGUAGAUCUGUUCGUCGUUGGUCUGGA) (SEQ ID NO: 9), CRI-47 (UAAUUUCUACUAAGUGUAGAUAGUAUUGCCUGUGUCGUCGG) (SEQ ID NO: 3, CRI-49 (UAAUUUCUACUAAGUGUAGAUUCGAUGUGUUGGCCUCGGUG) (SEQ ID NO: 10), CRI-90 (UAAUUUCUACUAAGUGUAGAUGACCGCUGGAGUGCUGGUGA) (SEQ ID NO: 11), and CRI-98 (UAAUUUCUACUAAGUGUAGAUCAAUUCCCCAGCUCACAAGC) (SEQ ID NO: 12) formed pool A. Guide RNAs CRI-41 (UAAUUUCUACUAAGUGUAGAUCGCGCCAAAGCGACAGCCAC) (SEQ ID NO: 1), CRI-74 (UAAUUUCUACUAAGUGUAGAUGUGCCGAUCGCGUUGGACGG)) (SEQ ID NO: 13), CRI-84 (UAAUUUCUACUAAGUGUAGAUAGGCGGUGCAGAUUGAUCGC) (SEQ ID NO: 14), CRI-93 (UAAUUUCUACUAAGUGUAGAUACUGCACACGUGGCCAGCAG) (SEQ ID NO: 15), and CRI-94 (UAAUUUCUACUAAGUGUAGAUGCUCACGCUGCUGCCGGAUA) (SEQ ID NO: 16) formed pool B. Guide RNAs CRI-58 (UAAUUUCUACUAAGUGUAGAUCCACAUGGGCGGCCACCGCU) (SEQ ID NO: 17), CRI-71 (UAAUUUCUACUAAGUGUAGAUCGCUAUUGGGGCCGCAGUGU) (SEQ ID NO: 18), CRI-73 (UAAUUUCUACUAAGUGUAGAUGGGGUCCGGUUCCGGCGGCC) (SEQ ID NO: 19), CRI-75 (UAAUUUCUACUAAGUGUAGAUGCCCACCGUGCCCGAUUGGU) (SEQ ID NO: 20), and CRI-77 (UAAUUUCUACUAAGUGUAGAUAUCUGCGCCACCUUGAAGUC) (SEQ ID NO: 21) formed pool C. Guide RNAs CRI-45 (UAAUUUCUACUAAGUGUAGAUGCUUGGUUGGACUUGACGAU) (SEQ ID NO: 2), CRI-57 (UAAUUUCUACUAAGUGUAGAUUUGGCGAUCGCGGUUCUGGC) (SEQ ID NO: 22), CRI-64 (UAAUUUCUACUAAGUGUAGAUCGGUAUGUGGCCUGUGACGG) (SEQ ID NO: 23), CRI-80 (UAAUUUCUACUAAGUGUAGAUUCUGCAGCCUGCCGUUCGAG) (SEQ ID NO: 24), and CRI-81 (UAAUUUCUACUAAGUGUAGAUUGCGUCAACACUGCGGAAUC) (SEQ ID NO: 25) formed pool D. Cas12a and guide pools were mixed and diluted to 2 nM for Cas12a and 0.5 nM for the guide pool in assay buffer prior to the experiment. dsDNA gBlock target was diluted to 400 fM and serially 3-fold diluted five times in assay buffer prior to the experiment. ssDNA soluble substrate (Biotin-CCCCCCCCCC-Alexa488) (SEQ ID NO: 26) synthesized by IDT was diluted to 40 nM in assay buffer prior to the experiment. Assay buffer composition was as follows: 10 mM Tris-HCl, pH 7.5, 10 mM MgCl2, 1 mM TCEP, 40 μg/mL BSA, and 0.01% Igepal CA-630. MyOne SA beads from Invitrogen (cat #65002) were diluted to 0.1 mg/mL in capture buffer (1 M NaCl, 10 mM Tris 7.5, 10 mM EDTA, 0.01% Tween-20) prior to use with soluble ssDNA substrate.

Assays were performed as follows. Cas12a and guide pools (A or A+B or A+B+C or A+B+C+D) were mixed together and incubated for 10 minutes at room temperature before the addition of target. Target dsDNA was added to Cas12a and guide pools and the reaction was further incubated for 50 minutes at 37° C. The reaction was initiated by adding substrate and was then incubated at 37° C. for 2 hours. The final concentrations in the assay were as follows: 1 nM Cas12a, 0.25 nM each guide RNA, 10 pM target DNA when present, 100 nM Alexa488 substrate. At the end of the reaction, magnetic beads were added to the reaction and incubated at room temperature for 15 minutes. Beads were then collected with a magnet and the supernatant was collected and assayed for fluorescence at 490/525 nm.

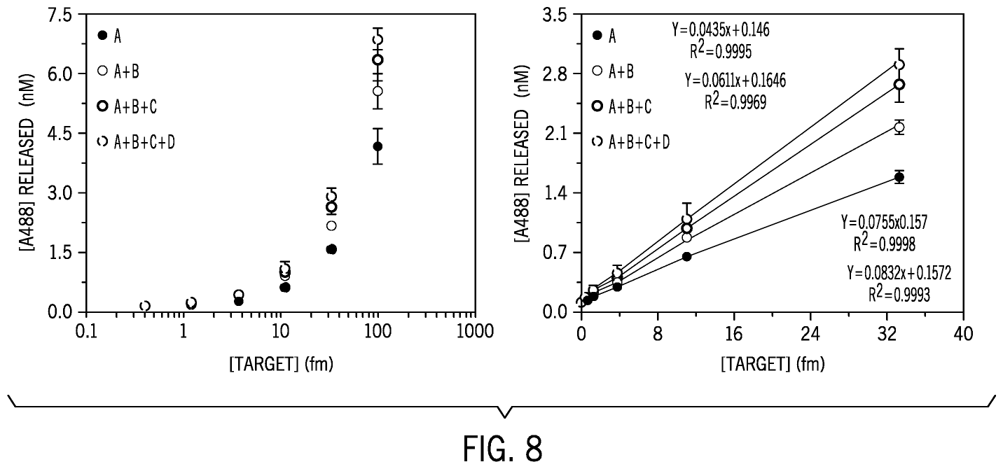

The results of this dog-piling experiment are shown in FIG. 8. As shown, the amount of reporter molecules released during the reaction increases as the number of guides used in the reaction increases. Such results demonstrate that “dog-piling” the guides results in better detection efficiency.

This disclosure has been made with reference to various example embodiments. However, those skilled in the art will recognize that changes and modifications can be made to the embodiments without departing from the scope of the present disclosure. For example, various operational steps, as well as components for carrying out operational steps, can be implemented in alternate ways depending upon the particular application or in consideration of any number of cost functions associated with the operation of the system e.g., one or more of the steps can be deleted, modified, or combined with other steps.

The herein described components (e.g., steps), devices, and objects and the discussion accompanying them are used as examples for the sake of conceptual clarity. Consequently, as used herein, the specific exemplars set forth and the accompanying discussion are intended to be representative of their more general classes. In general, use of any specific exemplar herein is also intended to be representative of its class, and the non-inclusion of such specific components (e.g., steps), devices, and objects herein should not be taken as indicating that limitation is desired.

With respect to the use of substantially any plural and/or singular terms herein, the reader can translate from the plural to the singular and/or from the singular to the plural as is appropriate to the context and/or application. The various singular/plural permutations are not expressly set forth herein for sake of clarity.

While particular aspects of the present subject matter described herein have been shown and described, it will be apparent to those skilled in the art that, based upon the teachings herein, changes and modifications can be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. In general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims can contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense the convention (e.g., “a system having at least one of A, B, or C” would include but not be limited to systems that have A alone, B alone, C alone, A and B together, A and C together, B and C together, and/or A, B, and C together, etc.). Virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While various aspects and embodiments have been disclosed herein, the various aspects and embodiments disclosed herein are for purposes of illustration and are not intended to be limiting, with the true scope and spirit being indicated by the following claims.

1. A device for determining a presence of a target nucleic acid sequence, the device comprising:
an assay area including a solution comprising an indicator device, wherein the indicator device comprises
at least one reporter molecule,
a tether molecule having,
a first end,
a second end, and
at least one indicator nucleic acid sequence for sensing the presence of an activated Cas nuclease positioned between the first and second ends, wherein the at least one reporter molecule is attached at the first end; and
a capture moiety attached at the second end of the tether molecule;
a detection area; and
a filter positioned between the detection area and the assay area.

2. The device of claim 1, wherein the at least one indicator nucleic acid sequence is greater than two nucleobases, including at least two bases selected from adenosine, uracil, or thymidine.

3. The device of claim 1, wherein the tether molecule includes at least one of polyethylene glycol (PEG), deoxyribonucleic acid (DNA), streptavidin, biotin, maleimide, sulfur, thiol, amino acids, proteins, succinimide, bacterial protein, haloalkane dehalogenase (HaloTag), chloroalkane, triazol, sulfone, glutamine, or lysine.

4. The device of claim 1, wherein the at least one reporter molecule includes one or more of a fluorescent molecule, luminescent molecule, a protein, a fusion protein, an enzyme, a SERS (surface enhanced Raman spectroscopy) particle, or a nanoparticle.

5. The device of claim 1, wherein the capture moiety is biotin.

6. The device of claim 1, wherein the assay area further comprises a modified Cas nuclease including a guide RNA sequence complementary to the target nucleic acid sequence.

7. The device of claim 1, wherein the assay area further comprises a biological sample.

8. The device of claim 1, wherein the assay area further comprises a capture moiety binding molecule that binds the capture moiety and sequesters tethered reporter molecules.

9. The device of claim 1, wherein the detection area comprises a capture molecule having an affinity for the reporter molecule.

10. The device of claim 1, wherein the Cas nuclease is Cas12.

11. The device of claim 10, wherein the Cas nuclease is Cas12a and the indicator nucleic acid sequence is single-stranded or double-stranded deoxyribonucleic acid.

12. The device of claim 1, wherein the Cas nuclease is Cas13.

13. The device of claim 12, wherein the Cas nuclease is Cas13a and the indicator nucleic acid sequence is single-stranded ribonucleic acid.

14. The device of claim 10, comprising a second modified Cas nuclease including a second guide RNA sequence complementary to a second target nucleic acid sequence.

15. The device of claim 14, comprising between 3 and 20 additional modified Cas nucleases, and guide RNA sequences complementary to between 3 and 20 additional target nucleic acid sequences.

16. A method of constructing a device for determining a presence of a target nucleic acid sequence, the method comprising:
synthesizing a tether molecule having,
a first end,
a second end, and
at least one indicator nucleic acid sequence positioned between the first and second end, the at least one indicator nucleic acid sequence including at least two nucleobases selected from two uracil bases and two thymidine bases;
attaching a soluble reporter molecule at the first end of the tether molecule; and
attaching a capture moiety at the second end of the tether molecule.

17. The method of claim 16, wherein the attaching of the capture moiety or the at least one reporter molecule includes covalently attaching one or more of a cysteine linkage or amine linkage.

18. The method of claim 16, wherein the indicator nucleic acid sequence is single-stranded ribonucleic acid.

19. The method of claim 16, wherein the indicator nucleic acid sequence is single-stranded or double-stranded deoxyribonucleic acid.

20. The method of claim 16, wherein the capture moiety is biotin.

21. A system for determining a presence of a target nucleic acid sequence, the system comprising:
a modified Cas nuclease including a guide RNA sequence complementary to the target nucleic acid sequence;
a solution comprising a device for determining a presence of an endonuclease, the device including;
at least one reporter molecule;
a tether molecule having,
a first end,
a second end, and
at least one indicator nucleic acid sequence positioned between the first and second end, wherein the at least one reporter molecule is attached at the first end; and
a capture moiety attached at the second end of the tether molecule;
an assay compartment;
a detection compartment; and
a filter positioned between the assay compartment and the detection compartment, wherein the filter is permeable to an untethered reporter molecule.

22. The system of claim 21, wherein at least one of the capture moiety or the at least one reporter molecule is covalently attached to the tether molecule by one or more of a cysteine linkage or amine linkage.

23. The system of claim 21, wherein the Cas nuclease is Cas12.

24. The system of claim 23, wherein the Cas nuclease is Cas12a and the indicator nucleic acid sequence is single-stranded or double-stranded deoxyribonucleic acid.

25. The system of claim 21, wherein the Cas nuclease is Cas13.

26. The system of claim 25, wherein the Cas nuclease is Cas13a and the indicator nucleic acid sequence is single-stranded ribonucleic acid.

27. The system of claim 21, comprising a second modified Cas nuclease including a second guide RNA sequence complementary to a second target nucleic acid sequence.

28. The system of claim 27, comprising between 3 and 20 additional modified Cas nucleases, and guide RNA sequences complementary to between 3 and 20 additional target nucleic acid sequences.

29. The system of claim 21, wherein the capture moiety is biotin.

30. A method of detecting a target nucleic acid sequence in a biological sample, the method comprising:
combining the biological sample with a composition to create a sample mixture, the composition including at least one modified Cas nuclease including a guide RNA having a sequence complementary to the target nucleic acid sequence;
incubating the sample mixture with a nuclease detection solution to create an assay solution, the nuclease detection solution including an indicator device comprising
a capture moiety;
at least one reporter molecule; and
a tether molecule having a first end and a second end, the tether molecule attached at a first end to the capture moiety and attached at the second end to the at least one reporter molecule, the tether molecule including at least one indicator sequence positioned between the first and second ends;
incubating the assay solution for an assay period;
applying a separating force to the assay solution; and
detecting a signal from an untethered reporter molecule, wherein if the detected signal is greater than a background value, the target sequence is present in the biological sample, wherein the background value is obtained from a biological sample lacking the target sequence.

31. The method of claim 30, wherein the biological sample is from a human and selected or derived from one or more of blood, sweat, serum, sputum, saliva, mucus, cells, or tissue.

32. The method of claim 30, wherein the target nucleic acid sequence is derived from a fungus, bacterium, virus, protozoa, or mammalian cell.

33. The method of claim 30, wherein the at least one reporter molecule is selected from one or more of a fluorescent molecule, a luminescent molecule, a fusion protein, a protein, an enzyme, a SERS particle, or a nanoparticle.

34. The method of claim 30, wherein the capture moiety is biotin.

35. The method of claim 30, wherein the tether molecule includes one or more of PEG, DNA, streptavidin, biotin, maleimide, sulfur, thiol, amino acids, proteins, succinimide, bacterial protein, haloalkane dehalogenase (HaloTag), chloroalkane, triazol, sulfone, glutamine, or lysine, and the tether molecule is covalently attached to the capture moiety and/or the reporter molecule.

36. The method of claim 30, wherein the separating force is selected from at least one of centrifugation, lateral fluid flow, microfluidic fluid flow, or magnetism.

37. The method of claim 30, wherein the signal is detected by one or more of Raman spectroscopy, fluorescence spectroscopy, luminometer, visual inspection, or surface plasmon resonance.

38. The method of claim 30, further comprising incubating the assay solution with a capture moiety binding molecule before applying the separating force to the assay solution.

39. The method of claim 30, further comprising filtering the untethered reporter molecule through a filter before detecting a signal from the untethered reporter molecule.

40. The method of claim 30, wherein detecting a signal from the untethered reporter molecule comprises contacting the assay solution with a plurality of capture molecules each having an affinity for the reporter molecule.

41. The method of claim 30, wherein the Cas nuclease is Cas12.

42. The method of claim 41, wherein the Cas nuclease is Cas12a and the at least one indicator sequence is single-stranded or double-stranded deoxyribonucleic acid.

43. The method of claim 30, wherein the Cas nuclease is Cas13.

44. The method of claim 43, wherein the Cas nuclease is Cas13a and the at least one indicator sequence is single-stranded ribonucleic acid.

45. The method of claim 30, comprising a second modified Cas nuclease including a second guide RNA sequence complementary to a second target nucleic acid sequence.

46. The method of claim 45, comprising between 3 and 20 additional modified Cas nucleases, and guide RNA sequences complementary to between 3 and 20 additional target nucleic acid sequences.

47. A method of detecting a target nucleic acid sequence in a biological sample, the method comprising:
obtaining a biological sample;
combining the biological sample with a composition comprising at least one Cas nuclease modified with a guide RNA sequence complementary to the target nucleic acid sequence, to create a sample mixture;
incubating the sample mixture with an indicator device in solution to create an assay solution, the indicator device including
a biotin molecule;
a tether molecule with a first end and a second end, wherein the first end is attached to the biotin molecule; and
at least one luciferase enzyme attached to a second end of the tether molecule, wherein the tether molecule includes at least one indicator nucleic acid sequence positioned between the first and second ends, and the at least one indicator nucleic acid sequence includes at least two nucleobases, including at least two uracil bases;
incubating the assay solution for an assay period;
incubating the assay solution with a biotin binding molecule to produce a detection solution;
applying a centrifugal force to the detection solution;
forcing at least a portion of the detection solution through a filter that is permeable to the at least one luciferase enzyme;
allowing an un-tethered luciferase enzyme molecule to pass through the filter into a detection compartment including luciferin;
detecting light produced responsive to oxidation of luciferin by the luciferase enzyme.

48. The method of claim 47, wherein the Cas nuclease is Cas12.

49. The method of claim 48, wherein the Cas nuclease is Cas12a and the at least one indicator nucleic acid sequence is single-stranded or double-stranded deoxyribonucleic acid.

50. The method of claim 47, wherein the Cas nuclease is Cas13.

51. The method of claim 50, wherein the Cas nuclease is Cas13a and the at least one indicator nucleic acid sequence is single-stranded ribonucleic acid.

52. A system for determining a presence of a target nucleic acid sequence, the system comprising:
a modified Cas nuclease, including a guide RNA sequence complementary to the target nucleic acid sequence;
an assay compartment comprising
a first reaction compartment comprising a tethered first molecule, and
a second reaction compartment comprising a solution including an indicator device comprising
a capture moiety;
at least one reporter molecule; and
a tether molecule having a first end and a second end, the tether molecule attached at a first end to the capture moiety and attached at the second end to the at least one reporter molecule, the tether molecule including at least one indicator sequence positioned between the first and second ends;
wherein the tethered first molecule cleaves the tether molecule of the indicator device in the second reaction compartment when the tethered first molecule is in an untethered state,
wherein cleavage of the tether molecule of the indicator device releases the at least one reporter molecule for detection;
a detection compartment for detecting the untethered reporter molecule; and
a filter positioned between the assay compartment and the detection compartment, wherein the filter is permeable to the untethered reporter molecule.

53. The system of claim 52, wherein the tethered first molecule is an enzyme.

54. The system of claim 53, wherein the enzyme is selected from a protease, a restriction enzyme, a nuclease, DNase, and RNase.

55. The system of claim 52, wherein the capture moiety is biotin.

56. The system of claim 52, wherein the at least one reporter molecule is selected from one or more of a fluorescent molecule, luminescent molecule, a protein, a fusion protein, an enzyme, a SERS (surface enhanced Raman spectroscopy) particle, or a nanoparticle.

57. The system of claim 52, wherein the detection compartment comprises a capture molecule having an affinity for the untethered reporter molecule.

58. The system of claim 52, wherein the Cas nuclease is Cas12.

59. The system of claim 58, wherein the Cas nuclease is Cas12a and the at least one indicator sequence is single-stranded or double-stranded deoxyribonucleic acid.

60. The system of claim 52, wherein the Cas nuclease is Cas13.

61. The system of claim 60, wherein the Cas nuclease is Cas13a and the at least one indicator sequence is single-stranded ribonucleic acid.

62. The system of claim 52, comprising a second modified Cas nuclease including a second guide RNA sequence complementary to a second target nucleic acid sequence.

63. The system of claim 62, comprising between 3 and 20 additional modified Cas nucleases, and guide RNA sequences complementary to between 3 and 20 additional target nucleic acid sequences.
